(19) United States
(12) Patent Application Publication
(10) Pub. No.: US 2023/0193406 A1
(43) Pub. Date: Jun. 22, 2023

(54) METHODS AND COMPOSITIONS FOR PROCESSING BOTANICAL MATERIALS

(71) Applicants: HERBALIFE INTERNATIONAL OF AMERICA, INC., Los Angeles, CA (US); The Chinese University of Hong Kong, Hong Kong (CN)

(72) Inventors: Zhengfei Lu, Alhambra, CA (US); Zhengxiu Yang, Shaoyang City (CN); Zheng Quan, Rancho Palos Verdes, CA (US); Pang-Chui Shaw, Hong Kong (CN); Yat-tung Lo, Hong Kong (CN)

(21) Appl. No.: 17/933,753

(22) Filed: Sep. 20, 2022

Related U.S. Application Data
(60) Provisional application No. 63/261,502, filed on Sep. 22, 2021.

(51) Int. Cl. C12Q 1/6895; C12Q 1/686; C12Q 1/6853

(52) U.S. Cl. CPC C12Q 1/6895; C12Q 1/686; C12Q 1/6853; C12Q 2600/16; C12Q 2600/156

(57) ABSTRACT

Some embodiments described herein are methods, systems, and kits using tetra-primer ARMS-PCR for identifying processed material and detecting adulterant in the material under a unified condition with high specificity and sensitivity. In some embodiments, the tetra-primer ARMS-PCR includes a pair of inner primers and a pair of outer primers, wherein one or both inner primers have a 5′ end random nucleic acid modification and/or a 3′ end phosphorothioate bond modification.

### CROSS-REFERENCE TO RELATED APPLICATIONS

This application claims the benefit of priority to U.S. Provisional Application No. 63/261,502, filed Sep. 22, 2021, the disclosure of which is hereby expressly incorporated by reference in its entirety.

### REFERENCE TO SEQUENCE LISTING

The present application is being filed along with a Sequence Listing in electronic format. The Sequence Listing is provided as a file HLIFE134ASEQUENCE.xml, created and last modified on Sep. 20, 2022, which is 41,276 bytes in size. The information in the electronic format of the Sequence Listing is incorporated herein by reference in its entirety.

### FIELD

The present disclosure relates to methods, systems, and kits for identifying processed material. In particular, the present disclosure relates to methods and kits for assessing botanical DNA fragments in dietary supplements.

### BACKGROUND

For botanical dietary supplement industry, botanical identification and adulteration prevention is the first step in botanical ingredient quality control. With multiple close species co-exist in commercial trade, one major form of adulteration is substituting the claimed species with another species. It is sometimes caused by mistakes made in the tracing documents or mismanagement of supply chain. More often, the adulteration is intentional due to economic incentive.

In the field, whole plant can be distinguished by the differences in their morphology features. However, typical commercial materials in industry supply chain contain only the plant parts and are often grounded into powders even processed into extracts, resulting the missing of essential morphological features for identification. Therefore, industry botanical identification often relies on analytical equipment. In many Pharmacopeias, chromatographic methods, such as Thin-Layer Chromatography (TLC) and High Performance Liquid Chromatography (HPLC) methods, have been incorporated for species identification based on the presence and absence of characteristic marker compounds and their ratios. However, sample's chemical profile subjects to variations, which could be caused by harvest time, geographic location, storage conditions and processing. In addition, the possibility of encountering mixed botanical materials in commerce adds another layer of uncertainty when interpreting chromatograms following methods that developed based on single botanical materials. To prevent unexpected outcomes due to adulteration, fit-for-purpose identification method must be used in the quality control of botanical ingredients.

### SUMMARY

Embodiments provided herein relate to methods for unifying tetra-primer ARMS-PCR conditions for botanical materials at various processing stages. In particular, the present disclosure relates to methods and kits for assessing botanical DNA fragments in dietary supplements. Also provided are systems and kits related to the same.

Some embodiments provided herein relate to methods for identifying processed botanical material. In some embodiments, the methods include extracting genomic plant DNA from the processed botanical material, wherein the processed botanical material contains a target species and an optional non-target species. In some embodiments, the methods include amplifying the extracted genomic plant DNA using tetra-primer amplification refractory mutation system polymerase chain reaction (ARMS-PCR). In some embodiments, the methods include identifying a PCR amplicon amplified from the target species and optionally another PCR amplicon amplified from the non-target species. In some embodiments, the methods include identifying the processed botanical material. In some embodiments, the methods further include detecting adulterant in the material.

In some embodiments, the botanical material is ginseng. In some embodiments, the ginseng is Panax ginseng, Panax quinquefolius, Panax notoginseng, Panax japonicas, or Eleutherococcus senticosus. In some embodiments, the botanical material is parsley or celery. In some embodiments, the parsley is Petroselinum crispum and wherein the celery is Apium graveolens. In some embodiments, the processed botanical material is a supplement, powder, or extract. In some embodiments, the tetra-primer ARMS-PCR includes a pair of inner primers and a pair of outer primers. In some embodiments, one or both inner primers of the pair of inner primers have a 5′ end random nucleic acid modification and/or a 3′ end phosphorothioate bond modification. In some embodiments, one or both inner primers of the pair of inner primers have 1-9 3′ end phosphorothioate bond modifications. In some embodiments, one or both inner primers of the pair of inner primers have 4 consecutive 3′ end phosphorothioate bond modifications. In some embodiments, the pair of inner primers and the pair of outer primers are present in a ratio of 1:1, 2:1, 3:1, 4:1, 5:1, 6:1, 7:1, 8:1, 9:1, 10:1, 11:1, 12:1, 13:1, 14:1, or 15:1. In some embodiments, the botanical material is ginseng, and the pair of inner primers comprises an inner forward primer having a sequence as set forth in SEQ ID NO: 12 and comprises an inner reverse primer having a sequence as set forth in SEQ ID NO: 13. In some embodiments, the botanical material is parsley, and the pair of inner primers comprises an inner forward primer having a sequence as set forth in SEQ ID NO: 31 or 33 and comprises an inner reverse primer having a sequence as set forth in SEQ ID NO: 32 or 34.

Some embodiments provided herein relate to multiplex PCR systems. In some embodiments, the systems are used for identifying processed botanical material. In some embodiments, the processed botanical material includes a target species and/or a closely related non-target species. In some embodiments, the systems include an inner forward primer and an inner reverse primer, wherein a 3′ terminus of the inner forward primer comprises a sequence that is complementary to a sequence specific to the target species, and wherein a 3′ terminus of the inner reverse primer comprises a sequence that is complementary to a sequence specific to the non-target species, or vice versa. In some embodiments, the systems include an outer primer pair consisting of an outer forward primer and an outer reverse primer.

In some embodiments, the sequence specific to the target species and the sequence specific to the non-target species differ by a single base or by a deletion. In some embodiments, the processed botanical material comprises an adulterant. In some embodiments, the inner forward primer and/or inner reverse primer have a 5′ end random nucleic acid modification, a 3′ end phosphorothioate bonds modification, or both. In some embodiments, the inner forward primer and/or inner reverse primer have 1-9 3′ end phosphorothioate bond modifications. In some embodiments, the inner forward primer and/or inner reverse primer have 4 consecutive 3′ end phosphorothioate bond modifications. In some embodiments, the systems further include a DNA polymerase that lacks 3′−>5′ exonuclease activity. In some embodiments, the DNA polymerase is a Taq DNA polymerase. In some embodiments, the inner primer and outer primer are in a ratio of 1:1, 2:1, 3:1, 4:1, 5:1, 6:1, 7:1, 8:1, 9:1, 10:1, 11:1, 12:1, 13:1, 14:1, or 15:1.

In some embodiments, the processed botanical material is a market ginseng root material. In some embodiments, the target species is P. ginseng. In some embodiments, the non-target species is P. quinquefolius, P. notoginseng, P. japonicus, or E. senticosus. In some embodiments, the inner forward primer comprises a sequence as set forth in SEQ ID NO: 12 and the inner reverse primer comprises a sequence as set forth in SEQ ID NO: 13.

In some embodiments, the target species is Petroselinum crispum. In some embodiments, the non-target species is Apium graveolens. In some embodiments, the inner forward primer comprises a sequence as set forth in SEQ ID NO: 31 or 33 and the inner reverse primer comprises a sequence as set forth in SEQ ID NO: 32 or 34.

Some embodiments provided herein relate to methods of identifying and differentiating a target species from a non-target species in a sample. In some embodiments, the methods include identifying a specific DNA region that differs by a single base or by a deletion between the target species and the non-target species. In some embodiments, the methods include providing an inner primer pair comprising an inner forward primer and an inner reverse primer, wherein a 3′ terminus of the inner forward primer is complementary to a sequence specific to the target species and a 3′ terminus of the inner reverse primer is complementary to a sequence specific to the non-target species, or vice versa, wherein the sequence specific to the target species and the sequence specific to the non-target species differ by a single base or by a deletion. In some embodiments, the methods include providing an outer primer pair comprising an outer forward primer and an outer reverse primer. In some embodiments, the methods include providing a sample comprising or suspected of comprising a target species and a non-target species. In some embodiments, the methods include performing a PCR reaction on the sample to identify the target species and/or non-target species in the sample. In some embodiments, the inner forward primer and/or inner reverse primers have a 5′ end random nucleic acid modification and/or a 3′ end phosphorothioate bond modification.

Some embodiments provided herein relate to multiplex PCR kits. In some embodiments, the kits are used for identifying and differentiating a target species from a non-target species in a sample. In some embodiments, the kits include an inner primer pair comprising an inner forward primer and an inner reverse primer, wherein a 3′ terminus of the inner forward primer is complementary to a sequence specific to the target species and a 3′ terminus of the inner reverse primer is complementary to a sequence specific to the non-target species, or vice versa, wherein the sequence specific to the target species and the sequence specific to the non-target species differ by a single base or by a deletion. In some embodiments, the kits include an outer primer pair consisting of an outer forward primer and an outer reverse primer. In some embodiments, the kits include a DNA polymerase that lacks 3′−>5′ exonuclease activity. In some embodiments, the DNA polymerase comprises a Taq DNA polymerase. In some embodiments, the inner forward and/or reverse primers have a 5′ end random nucleic acid modification and/or a 3′ end phosphorothioate bond modification.

### BRIEF DESCRIPTION OF THE DRAWINGS

FIG. 1 depicts a schematic representation of the methods provided herein for using tetra-primer ARMS-PCR to differentiate various botanical species. The left panel depicts typical ARMS-PCR methods, whereas the right panel depicts modified ARMS-PCR using the methods described herein ,resulting in increased sensitivity for differentiating between target and adulterant.

FIG. 2 depicts locations and sequences of outer and inner primers used in Tetra-primer ARMS-PCR for rapid P. ginseng identification and adulteration detection.

FIG. 3 depicts a schematic diagram of rapid P. ginseng identification and adulteration detection assay result.

FIG. 4 depicts a schematic diagram of rapid P. ginseng identification and adulteration detection assay result on DNA visualization equipment.

FIG. 5 depicts rapid P. ginseng identification and adulteration detection assay yields different patterns in P. ginseng and other ginseng botanical reference materials at low PCR cycles (28 cycles).

FIG. 6 depicts application of rapid P. ginseng identification and adulteration detection assay in ginseng materials at different processing stages across different PCR cycle numbers shown reduced sensitivity and specificity. Reduced sensitivity is shown by the reduction of band intensity at high PCR cycles (35 and 40 cycles). Reduced specificity is shown by non-specific amplification of other diagnostic bands (arrows) in red ginseng sample at high PCR cycles.

FIG. 7 depicts altering outer and inner primer ratio in the rapid P. ginseng identification and adulteration detection assay based on prior publication improves sensitivity but further reduces specificity (middle and right arrows). Left most arrow shown non-specific amplification resulting from high inner vs. outer primer ratio.

FIG. 8 depicts introducing additional 3′ end terminal mismatch along with altering outer and inner primer ratio in the rapid P. ginseng identification and adulteration detection assay based on prior publication reduces non-specificity products but not completely blocking non-specific amplification (middle and right arrows). Left most arrow shown non-specific amplification resulting from high inner vs. outer primer ratio. The mismatches are underlined and located at the -2 position from 3′ terminus of inner primers.

FIG. 9 depicts schematic mechanism diagram to illustrate reduced sensitivity in original rapid P. ginseng identification and adulteration detection assay.

FIG. 10 depicts schematic mechanism diagram to illustrate sensitivity improvement in rapid P. ginseng identification and adulteration detection assay when replacing original inner primers with inner primers with random nucleotides added the 5′ end terminal.

FIG. 11 depicts comparison between rapid P. ginseng identification and adulteration detection assay using original inner primers and inner primers with random nucleotides added the 5′ end terminal on multiple ginseng reference materials across different PCR cycle numbers. Left panel: PCR results using regular inner primer set; right panel: PCR results using inner primer set with a 5′ terminus random nucleotide.

FIG. 12 depicts target vs. control band molar ratio statistical analysis between rapid P. ginseng identification and adulteration detection assay using original inner primers and inner primers with random nucleotides added the 5′ end terminal.

FIG. 13 depicts performance of rapid P. ginseng identification and adulteration detection assay on reference materials using modifications other than 5′ end terminal random nucleotides.

FIG. 14 depicts performance of rapid P. ginseng identification and adulteration detection assay on reference materials using the 5′ end terminal random nucleotides and 3′ end terminal base mismatch or phosphorothioate bonds to improve assay specificity at high PCR cycle numbers.

FIG. 15 depicts rapid P. ginseng identification and adulteration detection assay with inner primer 5′ end terminal random nucleotides and 3′ end phosphorothioate bonds modification yields patterns in P. ginseng and other ginseng botanical reference materials at high PCR cycles.

FIG. 16 depicts rapid P. ginseng identification and adulteration detection assay with inner primer modification (both 3′ end phosphorothioate bonds modification and 5′ end random nucleotide) yields patterns in P. ginseng, other ginseng botanical reference materials, and processed materials (steamed roots and extracts) under unified condition.

FIG. 17 depicts performance of rapid P. ginseng identification and adulteration detection assay with inner primer modification in unprocessed or lightly processed market ginseng materials.

FIG. 18 depicts performance of rapid P. ginseng identification and adulteration detection assay with inner primer modification in highly processed market ginseng materials, including P. ginseng extracts, steamed roots (red ginseng), and decoctions.

FIG. 19 depicts retest single red ginseng root slice using rapid P. ginseng identification and adulteration detection assay with inner primer modification.

FIG. 20 depicts performance of rapid P. ginseng identification and adulteration detection assay with inner primer modification in P. quinquefolius and P. ginseng root admixture at various ratio. The data shows that P. quinquefolius adulteration in P. ginseng can be consistently detected using capillary electrophoresis when w/w ratio reaches 10%/90%.

FIG. 21 depicts performance of rapid P. ginseng identification and adulteration detection assay with inner primer modification in P. quinquefolius and P. ginseng root admixture at various ratio by agarose gel electrophoresis. The data shows that P. quinquefolius adulteration in P. ginseng can also be detected using regular gel electrophoresis.

FIG. 22 depicts performance of rapid P. ginseng identification and adulteration detection assay with inner primer modification in P. notoginseng, P. japonicus, E. senticosus and P. ginseng root admixture at various ratio. The data shows that detection of P. notoginseng, P. japonicus, and E. senticosus adulteration in P. ginseng were achieved at 50%, 50%, 40% (w/w).

FIG. 23 depicts performance of rapid P. ginseng identification and adulteration detection assay with inner primer modification in decoctions prepared from P. quinquefolius, P. notoginseng, P. japonicus, E. senticosus and P. ginseng root admixture at various ratio. The data shows that current assay reaches 5% (w/w) detection sensitivity in mixed materials after made into decoction.

FIG. 24 depicts locations and sequences of outer and inner primers used in Tetra-primer ARMS-PCR for rapid P. crispum identification and A. graveolens adulteration detection.

FIG. 25 depicts a schematic diagram of rapid P. crispum identification and A. graveolens adulteration detection assay result.

FIG. 26 depicts rapid P. crispum identification and A. graveolens adulteration detection assay yields different patterns in P. crispum (62 bp) and A. graveolens (193 bp) botanical reference materials at low PCR cycles (25 and 28 cycles).

FIG. 27 depicts application of rapid P. crispum identification and A. graveolens adulteration detection assay in parsley extracts at low PCR cycles (28 cycles).

FIG. 28 depicts application of rapid P. crispum identification and A. graveolens adulteration detection assay in parsley extracts at high PCR cycles (40 cycles). Arrows indicates non-specific amplifications.

FIG. 29 depicts application of rapid P. crispum identification and A. graveolens adulteration detection assay in parsley extracts at high PCR cycles (40 cycles).

FIG. 30 depicts application of rapid P. crispum identification and A. graveolens adulteration detection assay in parsley extracts at high PCR cycles (40 cycles).

FIG. 31 depicts locations and sequences of new outer primer and inner primers used in Tetra-primer ARMS-PCR for rapid P. crispum identification and A. graveolens adulteration detection.

FIG. 32 depicts a schematic diagram of rapid P. crispum identification and A. graveolens adulteration detection assay result.

FIG. 33 depicts rapid P. crispum identification and A. graveolens adulteration detection assay yields different patterns in P. crispum (113 bp) and A. graveolens (163 bp) botanical reference materials at low PCR cycles (25 and 28 cycles).

FIG. 34 depicts application of rapid P. crispum identification and A. graveolens adulteration detection assay in parsley extracts at low PCR cycles (28 cycles).

FIG. 35 depicts application of rapid P. crispum identification and A. graveolens adulteration detection assay in parsley extracts at high PCR cycles (40 cycles). Arrows indicates non-specific amplifications.

FIG. 36 depicts application of rapid P. crispum identification and A. graveolens adulteration detection assay in parsley extracts at high PCR cycles (40 cycles).

FIG. 37 depicts application of rapid P. crispum identification and A. graveolens adulteration detection assay in parsley extracts at high PCR cycles (40 cycles).

### DETAILED DESCRIPTION

In the Summary Section above and the Detailed Description Section, and the claims below, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there is a plurality of definitions for a term herein, those in this section prevail unless stated otherwise.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ including but not limited to,' or the like; the term ‘comprising’ as used herein is synonymous with ‘including,’ containing,' or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ preferred,“desired,” or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps but may include additional steps. When used in the context of a compound, composition or device, the term “comprising” means that the compound, composition, or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

“Sample” or “biological sample” is meant any material derived from a living or dead organism. The sample may be treated to physically, chemically and/or mechanically disrupt tissue or cell structure, thus releasing intracellular components. Sample preparation may use a solution that contains buffers, salts, enzymes, detergents and the like which are used to prepare the sample for analysis. Samples may be pooled from two or more sources. Samples may be fractionated.

“Nucleic acid” refers to a multimeric compound comprising two or more covalently bonded nucleosides or nucleoside analogs made up of a sugar moiety and a nitrogenous heterocyclic bases, or base analogs. Nucleosides are linked together by phosphodiester bonds or other linkages to form RNA, DNA, or chimeric DNA-RNA polymers or oligonucleotides, and analogs thereof. A nucleic acid “backbone” may be made up of a variety of linkages, (see, e.g., International Patent Application Pub. No. WO 95/32305). The sugar moiety of one or more residues in the nucleic acid may be either ribose or deoxyribose, or similar compounds having known substitutions such as, for example, 2′-methoxy substitutions and 2′-halide substitutions (e.g., 2′-F). The nitrogenous base of one or more residues in the nucleic acid may be conventional bases (A, G, C, T, U), analogs thereof (see, e.g., The Biochemistry of the Nucleic Acids 5-36, Adams et al., ed., 11th ed., 1992; Abraham et al., 2007, BioTechniques 43: 617-24), which include derivatives of purine or pyrimidine bases (see e.g., U.S. Pat. Nos. 5,378,825, 6,949,367 and International Patent Application Pub. No. WO 93/13121), or “abasic” wherein the nucleoside unit is lacking a nitrogenous base (see, e.g., U.S. Pat. No. 5,585,481). Nucleic acids may include one or more “locked nucleic acid” (LNA) residues (Vester et al., Biochemistry 43:13233-41, 2004). Nucleic acids may include a 3′-terminal dideoxynucleotide to block additional nucleotides from being added to the nucleic acid. Synthetic methods for making nucleic acids in vitro are well known in the art although nucleic acids may be purified from natural sources using routine techniques. The backbone of an oligomer may affect stability of a hybridization complex (e.g., formed between of a capture oligomer to its target nucleic acid). Such embodiments include peptide linkages, 2′-0-methoxy linkages and sugar-phosphodiester type linkages. Peptide nucleic acids are advantageous for forming a hybridization complex with RNA. An oligomer having 2′-methoxy substituted RNA groups or a 2′-fluoro substituted RNA may have enhance hybridization complex stability relative to standard DNA or RNA and is preferred for forming a hybridization complex with a complementary 2′-OH RNA. A linkage joining two sugar groups may affect hybridization complex stability by affecting the overall charge or the charge density, or by affecting steric interactions (e.g., bulky linkages may reduce hybridization complex stability). Preferred linkages include those with neutral groups (e.g., methylphosphonates) or charged groups (e.g., phosphorothioates) to affect complex stability.

As used herein, the term “hybridizing”, “hybridize”, “hybridization”, “annealing”, or “anneal” are used interchangeably in reference to the pairing of complementary nucleic acids using any process by which a strand of nucleic acid joins with a complementary strand through base pairing to form a hybridization complex. Hybridization and the strength of hybridization (for example, the strength of the association between the nucleic acids) is impacted by such factors as the degree of complementarity between the nucleic acids, stringency of the conditions involved, the melting temperature (Tm) of the formed hybrid, and the G:C ratio within the nucleic acids.

A “nucleotide” as used herein is a subunit of a nucleic acid consisting of a phosphate group, a 5-carbon sugar, and a nitrogenous base. The 5-carbon sugar found in RNA is ribose. In DNA, the 5-carbon sugar is 2′-deoxyribose. The term also includes analogs of such subunits, such as a methoxy group at the 2′ position of the ribose (2′-O-Me). As used herein, methoxy oligonucleotides containing “T” residues have a methoxy group at the 2′ position of the ribose moiety, and an uracil at the base position of the nucleotide.

A “target nucleic acid” as used herein is a nucleic acid comprising a “target sequence” to be amplified. Target nucleic acids may be DNA or RNA as described herein and may be either single-stranded or double-stranded. The target nucleic acid may include other sequences besides the target sequence, which may not be amplified. Target nucleic acids include the genomic nucleic acid, a gene product (e.g., mRNA), and amplification products thereof.

The term “target sequence” as used herein refers to the particular nucleotide sequence of the target nucleic acid that is to be amplified and/or detected. The “target sequence” includes the complexing sequences to which oligonucleotides (e.g., priming oligonucleotides and/or promoter oligonucleotides) complex during the processes of amplification. Where the target nucleic acid is originally single-stranded, the term “target sequence” will also refer to the sequence complementary to the “target sequence” as present in the target nucleic acid. Where the target nucleic acid is originally double-stranded, the term “target sequence” refers to both the sense (+) and antisense (−) strands.

The term “region” as used herein refers to a portion of a nucleic acid wherein said portion is smaller than the entire nucleic acid. For example, when the nucleic acid is a plant genome, the term “region” may be used to refer to a smaller area of the nucleic acid, wherein the smaller area is targeted by one or more oligonucleotides of the invention. The target binding sequence of an oligonucleotide may hybridize all or a portion of a region. A target binding sequence that hybridizes to a portion of a region is one that hybridizes within the referenced region.

By “complementary” is meant that the nucleotide sequences of similar regions of two single-stranded nucleic acids, or to different regions of the same single-stranded nucleic acid have a nucleotide base composition that allow the single-stranded regions to hybridize together in a stable double-stranded hydrogen-bonded region under stringent hybridization or amplification conditions. Sequences that hybridize to each other may be completely complementary or partially complementary to the intended target sequence by standard nucleic acid base pairing (e.g. G:C, A:T or A:U pairing). By “sufficiently complementary” is meant a contiguous sequence that is capable of hybridizing to another sequence by hydrogen bonding between a series of complementary bases, which may be complementary at each position in the sequence by standard base pairing or may contain one or more non-complementary residues, including abasic residues. Sufficiently complementary contiguous sequences typically are at least 80%, or at least 90%, complementary to a sequence to which an oligomer is intended to specifically hybridize (including all whole and rational numbers up to and including 100%). Sequences that are “sufficiently complementary” allow stable hybridization of a nucleic acid oligomer with its target sequence under appropriate hybridization conditions, even if the sequences are not completely complementary. When a contiguous sequence of nucleotides of one single-stranded region is able to form a series of “canonical” hydrogen-bonded base pairs with an analogous sequence of nucleotides of the other single-stranded region, such that A is paired with U or T and C is paired with G, the nucleotides sequences are “completely” complementary, (e.g., Sambrook et al., Molecular Cloning, A Laboratory Manual, 2nd ed. (Cold Spring Harbor Laboratory Press, Cold Spring Harbor, NY, 1989) at §§ 1.90-1.91, 7.37-7.57, 9.47-9.51 and 11.47-11.57, particularly §§ 9.50-9.51, 11.12-11.13, 11.45-11.47 and 11.55-11.57).

The interchangeable terms “oligomer,” “oligo,” and “oligonucleotide” refer to a polynucleotide having a contiguous nucleotide residue (nt) length of from 1,000 nts to as few as 5 nts. It is understood that the range from 1000 to as few as 5 is an inclusive range such that 1000 nts, 5 nts and each whole number of nts there between are included in the range. Oligonucleotides may be purified from naturally occurring sources or may be synthesized using any of a variety of well-known enzymatic or chemical methods. The term oligonucleotide does not denote any particular function to the reagent; rather, it is used generically to cover all such reagents described herein.

Amplification oligomers may be referred to as “primers.” A “primer” refers to an oligonucleotide that hybridizes to a template nucleic acid and has a 3′ end that can be extended in a known polymerization reaction. The 5′ region of the primer may be non-complementary to the target nucleic acid. In some embodiments, blocking moieties replace an oligomer's 3′0H to prevent enzyme-mediated extension of the oligomer in an amplification reaction. In alternative embodiments a blocking moiety may be within five residues of the 3′ end and is sufficiently large to limit binding of a polymerase to the oligomer. In other embodiments a blocking moiety is covalently attached to the 3′ terminus of an oligomer. Many different chemical groups may be used to block the 3′ end of an oligomer, including, but not limited to, alkyl groups, non-nucleotide linkers, alkane-diol dideoxynucleotide residues, and cordycepin. Those skilled in the art will further appreciate that any oligomer that can function as a primer (i.e., an amplification oligonucleotide that hybridizes specifically to a target sequence and has a 3′ end that can be extended by a polymerase).

Amplification of a “fragment” or “portion” of the target sequence refers to production of an amplified nucleic acid containing less than the entire target region nucleic acid sequence. Such fragments may be produced by amplifying a portion of the target sequence, e.g., by using an amplification oligonucleotide that hybridizes to and initiates polymerization from an internal position in the target sequence.

The term “amplicon” or the term “amplification product” as used herein refers to the nucleic acid molecule generated during an amplification procedure that is complementary or homologous to a sequence contained within the target sequence. This complementary or homologous sequence of an amplicon is sometimes referred to herein as a “target-specific sequence.” Amplicons can be double stranded or single stranded and can include DNA, RNA, or both. For example, DNA-dependent RNA polymerase transcribes single stranded amplicons from double stranded DNA during transcription-mediated amplification procedures. These single stranded amplicons are RNA amplicons and can be either strand of a double stranded complex; depending on how the amplification oligomers are designed. Thus, amplicons can be single stranded RNA. RNA-dependent DNA polymerases synthesize a DNA strand that is complementary to an RNA template. Thus, amplicons can be double stranded DNA and RNA hybrids. RNA-dependent DNA polymerases often include RNase activity, or are used in conjunction with an RNase, which degrades the RNA strand. Thus, amplicons can be single stranded DNA. RNA-dependent DNA polymerases and DNA-dependent DNA polymerases synthesize complementary DNA strands from DNA templates. Thus, amplicons can be double stranded DNA. RNA-dependent RNA polymerases synthesize RNA from an RNA template. Thus, amplicons can be double stranded RNA. DNA Dependent RNA polymerases synthesize RNA from double stranded DNA templates, also referred to as transcription. Thus, amplicons can be single stranded RNA. Amplicons and methods for generating amplicons are known to those skilled in the art. For convenience herein, a single strand of RNA or a single strand of DNA may represent an amplicon generated by an amplification oligomer combination of the current invention. Such representation is not meant to limit the amplicon to the representation shown. Skilled artisans in possession of the instant disclosure will use amplification oligomers and polymerase enzymes to generate any of the numerous types of amplicons; all within the spirit of the current invention.

By “amplification oligonucleotide” or “amplification oligomer” is meant an oligonucleotide, at least the 3′-end of which is complementary to a target nucleic acid, and which hybridizes to a target nucleic acid, or its complement, and participates in nucleic acid amplification. Examples of amplification oligomers include primers. Preferably, an amplification oligonucleotide contains at least 10 contiguous bases, and more preferably at least about 12 contiguous bases but less than about 70 bases, that hybridize specifically with a region of the target nucleic acid sequence under standard hybridization conditions. The contiguous bases that hybridize to the target sequence are at least about 80%, preferably at least about 90%, and more preferably about 100% complementary to the sequence to which the amplification oligonucleotide hybridizes. At least about X% refers to all a range of all whole and partial numbers from X% to 100%. An amplification oligonucleotide optionally may include modified nucleotides.

“Amplification” refers to any known procedure for obtaining multiple copies of a target nucleic acid sequence or its complement or fragments thereof, and preferred embodiments amplify the target specifically by using sequence-specific methods. Known amplification methods include, for example, transcription-mediated amplification, replicase-mediated amplification, polymerase chain reaction (PCR) amplification, including RT-PCR, ligase chain reaction (LCR) amplification and strand-displacement amplification (SDA). Replicase-mediated amplification uses self-replicating RNA molecules, and a replicase such as QB-replicase (e.g., see U.S. Patent No. 4,786,600 to Kramer et al. and PCT No. WO 90/14439). PCR amplification is well known and uses DNA polymerase, sequence-specific primers, and thermal cycling to synthesize multiple copies of the two complementary strands of DNA or cDNA (e.g., U.S. Pat. Nos. 4,683,195, 4,683,202, and 4,800,159 to Mullis et al., and Methods in Enzymology, 1987, Vol. 155: 335-350). LCR amplification uses at least four separate oligonucleotides to amplify a target and its complementary strand by using multiple cycles of hybridization, ligation, and denaturation (EP Patent No. 0 320 308). SDA amplifies by using a primer that contains a recognition site for a restriction endonuclease which nicks one strand of a hemi modified DNA duplex that includes the target sequence, followed by amplification in a series of primer extension and strand displacement steps (U.S. Pat. No. 5,422,252 to Walker et al.) It will be apparent to one skilled in the art that method steps and amplification oligonucleotides of the present invention may be readily adapted to a variety of nucleic acid amplification procedures based on primer extension by a polymerase activity.

The term “specificity,” in the context of an amplification and/or detection system, is used herein to refer to the characteristic of the system which describes its ability to distinguish between target and non-target sequences dependent on sequence and assay conditions. In terms of nucleic acid amplification, specificity generally refers to the ratio of the number of specific amplicons produced to the number of side-products (e.g., the signal-to-noise ratio). In terms of detection, specificity generally refers to the ratio of signal produced from target nucleic acids to signal produced from non-target nucleic acids.

The term “sensitivity” is used herein to refer to the precision with which a nucleic acid amplification reaction can be detected or quantitated. The sensitivity of an amplification reaction is generally a measure of the smallest copy number of the target nucleic acid that can be reliably detected in the amplification system, and will depend, for example, on the detection assay being employed, and the specificity of the amplification reaction, e.g., the ratio of specific amplicons to side-products.

Recently, genomic testing is rising as an alternative analytical approach to provide species information in botanical quality control. In contrast to chromatographic methods, genomic identification methods utilize characteristic nucleotide sequence, which is specific at species level and not subject to change due to environmental factors. The species difference in nucleotide sequences can be assessed by either sequencing or specific amplification. Although sequencing is the gold standard to confirm the sequence difference between the diagnostic regions, it involves high capital investment and requires additional steps after amplification of the diagnostic regions. Species-specific PCR amplification is another option, a well-designed primer pair can produce amplicon in species specific manner. Without the additional sequencing step, species-specific PCR requires low capital investment, have shorter turnaround time, and suitable for routine testing.

DNA-based molecular analysis techniques, such as randomly amplified polymorphic DNA (RAPD), PCR-restriction fragment length polymorphism (PCR-RFLP) analyses, hybridization, microarrays, and DNA barcoding have been introduced to botanical authentication to complement the traditional physical and chemical identification methods (Heubl, G. (2010). Planta Med, 76(17), 1963-1974, incorporated by reference herein in its entirety). Traditional botanical identification methods include morphological and chemical methods. However, these methods are challenged by the loss of physical features in the processed product and variation in botanical chemical profiles due to seasonal and geographical differences. DNA barcodes provide a relative stable profile for identification purposes. A single barcode for botanical identification is yet to be found. Using multiple DNA barcodes together will increase their discrimination power. However, 3 or more barcodes do not provide additional discrimination power than 2 barcodes if the appropriate combination is chosen (C. P. W. Group. PNAS 106(31), (2009), pp. 12794-12797; incorporated herein by reference in its entirety). Two-tiered DNA barcoding has been applied to botanical identification in research, but industry regulations require a validated method and a defined scope (Newmaster et al. BMC medicine 11.1 (2013), p. 222; Pawar et al. Planta Medial 82.05 (2016), 0A17; each of which is incorporated herein by reference in its entirety).

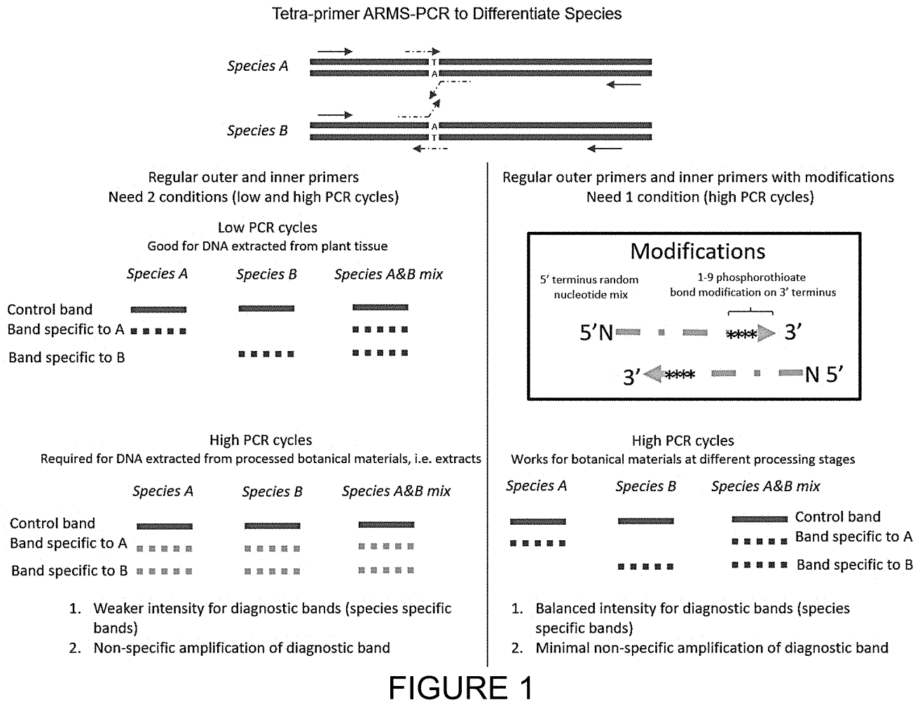

Tetra-primer ARMS-PCR is a simple and economical tool for botanical quality control. However, current methods require different assay conditions for botanical materials at different processing stages. In addition, at high amplification cycle numbers, false positive and false negative incidences caused by Taq DNA polymerase might cause botanical misidentification, which means rejection of the botanical material of correct species or acceptance of wrong botanical material for manufacture. The current invention improves the applicability of original Tetra-primer ARMS-PCR method in all types of botanical materials, so the evaluation can be done under a unified condition with high specificity and sensitivity. A unified assay for botanical materials at all processing stages will significantly improve the efficiency of routine botanical material quality control practice, as shown in FIG. 1.

Some embodiments relate to a method for identifying processed botanical material and optionally detecting adulterant in the material, the method comprises: i) extracting genomic plant DNA from the processed botanical material, wherein the processed botanical material contains a target species and an optional non-target species; ii) amplifying the extracted genomic plant DNA using tetra-primer amplification refractory mutation system polymerase chain reaction (ARMS-PCR); iii) identifying a PCR amplicon amplified from the target species and optionally another PCR amplicon amplified from the non-target species; iv) thereby identifying the processed botanical material and optionally detecting adulterant in the material.

In some embodiments disclosed herein, the processed botanical material is a supplement, powder, or extract. In some embodiments, the processed botanical material has been treated with high temperature or extraction.

In some embodiments, the botanical material may be a nutraceutical composition or a dietary supplement that includes a botanical matter, a processed botanical extract, or a botanical powder, including a sterilized botanical powder. As used herein an “extract” or “botanical extract” refers to a solid, viscid, or liquid substance or preparation that includes a substance of plant, such as a root, a leaf, a stem, a flower, a seed, a fruit, or other portion of a plant. In some embodiments, the botanical material is a raw material, a powder, or an extract. As used herein the term “processed” includes treatment of a botanical substance to develop an herbal medicine, a nutraceutical composition, or a dietary supplement, including grinding, heating, fermenting, compacting, degrading, drying, wetting, or otherwise processing the botanical substance for preparation of the end botanical product for use or consumption by a consumer.

The term botanical pertains to or relates to plants. The term “plant,” includes plants and plant parts including but not limited to plant cells and plant tissues such as leaves, stems, roots, flowers, pollen, fruit, bark, and seeds. The class of plants that can be used in the present invention is generally as broad as the class of higher and lower plants that may be commonly used in herbal medicines or in dietary supplements to provide a therapeutic or aesthetic benefit. In some embodiments, any botanical of interest may be used. For example, a botanical that has a closely related adulterant, and for which one wishes to distinguish between the botanical and the adulterant may be used. In some embodiments, a botanical includes chamomile (including Matricaria chamomilla (also referred to as German chamomile or Matricaria recutita), feverfew (Tanacetum parthenium), Roman chamomile (Chamaemelum nobile syn anthemis nobilis), Chinese chamomile (Chrysanthemum x morifolium, or Chrysanthemum indicum), guarana (Paullinia cupana), parsley (Petroselinum crispum), celery (Apium graveolens), fennel (Foeniculum vulgare), Asian ginseng (Panax ginseng), American ginseng (Panax quinquefolius), Tienchi ginseng (Panax notoginseng), Siberian ginseng (Eleutherococcus senticosus) Dong Quai (Angelica sinensis), garden angelica (Angelica archangelica), pubescent angelica (Angelica pubescens), dahurian angelica (Angelica dahurica), Chinese cinnamon (Cinnamomum cassia), true cinnamon (Cinnamomum verum syn Cinnamomum zeylanicum), Indonesian cinnamon (Cinnamomum burmannii), Ginkgo (Ginkgo biloba), Japanese sophora (Sophora japonica), buckwheat (Fagopyrum esculentum), jujube (Ziziphus spinosa), Indian jujube (Ziziphus mauritiana), Japanese raisin tree (Hovenia dukis), ginger (Zingiber officinale), lesser galangal (Alpinia officinarum), greater galangal (Alpinia galanga), schisandra (Schisandra chinensis), southern schisandra (Schisandra sphenanthera), astragalus (Astragalus membranaceus), maca (Lepidium meyenii), radish (Raphanus sativus), turnip (Brassica rapa), peppermint (Mentha piperita), Chinese mint (Mentha canadensis), green tea (Camellia sinensis), rosemary (Rosmarinus officinalis), bilberry (Vaccinium myrtillus), blueberry (Vaccinium corymbosum), cranberry (Vaccinium macrocarpon), mulberry (Monts alba), or guarana (Paullinia cupana).

In some embodiments disclosed herein, the botanical material is ginseng (for example, Panax ginseng, Panax quinquefolius, Panax notoginseng, Panax japonicas, Eleutherococcus senticosus).

In some embodiments, the botanical material is parsley.

As used herein, the term “genomic DNA” refers to the chromosomal DNA sequence of a gene or segment of a gene, including the DNA sequences of non-coding as well as coding regions. Genomic DNA also refers to DNA isolated directly from cells or chromosomes or the cloned copies of all or part of such DNA. In some embodiments, the isolated genomic DNA is isolated from a processed botanical sample, such that the sample includes botanical DNA fragments. As used herein, fragmented DNA refers to portions of DNA having less than about 300 bp due to the processing of the botanical material, such as about 300 bp, 290 bp, 280 bp, 270 bp, 260 bp, 250 bp, 240 bp, 230 bp, 220 bp, 210 bp, 200 bp, 190 bp, 180 bp, 170 bp, 160 bp, 150 bp, 140 bp, 130 bp, 120 bp, 110 bp, 100 bp, 90 bp, 80 bp, 70 bp, 60 bp, 50 bp, 40 bp, 30 bp, 20, or 10 bp, or within a range defined by any two of the aforementioned values.

As described herein, botanical DNA fragments are present in dietary supplements in low quantity or low quality, or both, and therefore, are unable to be readily detected by conventional techniques. For example, in some embodiments, the botanical DNA fragments may be excessively degraded or sufficiently fragmented as to be incapable of being detected. In addition, in some embodiments, a target botanical DNA fragments may be present in a botanical product (for example, an herbal medicine, a nutraceutical composition, or a dietary supplement) in an amount of about 100 ng, 10 ng, 1 ng, 900 pg, 800 pg, 700 pg, 600 pg, 500 pg, 400 pg, 300 pg, 200 pg, 100 pg, 10 pg, 1 pg, 900 fg, 800 fg, 700 fg, 600 fg, 500 fg, 400 fg, 300 fg, 200 fg, 100 fg, or less, or an amount within a range defined by any two of the aforementioned values. In this way, botanical DNA fragments in processed botanical materials are sometimes referred to as “invisible,” referring to the inability to visualize or detect the fragments. As used herein the term “detection” or “visualization” refers to the ability to observe DNA fragments. The detection of the fragments allows for downstream analysis. Detection of the fragments can be performed by DNA detection techniques, including by Southern blot, the analysis of DNA on agarose or acrylamide gels to fractionate the DNA according to size, which may be followed by transfer and immobilization of the DNA from the gel to a solid support, such as nitrocellulose or a nylon membrane. The immobilized DNA may further be probed with a labeled oligodeoxyribonucleotide probe or DNA probe to detect DNA species complementary to the probe used. The DNA may be cleaved with restriction enzymes prior to electrophoresis. Following electrophoresis, the DNA may be partially depurinated and denatured prior to or during transfer to the solid support.

Adulterant refers to unwanted substances in the processed botanical material. Adulterant can be added to the processed botanical material accidently, negligently, or intentionally.

The methods for extracting genomic plant DNA are generally known in the art. Some methods involve physical grinding of cells or tissue followed by extraction in buffers containing detergent, EDTA, Tris and other reagents. Some methods use a solid phase extraction material comprising silica and having hydroxyl groups on its surface to replace phenol for removal of proteins. Some methods for extracting genomic DNA (gDNA) from plants employs cetyltrimethylammonium bromide (CTAB) to precipitate nucleic acids and acidic polysaccharides from solutions of low ionic strength but can also be used to remove polysaccharides and proteins from solutions of higher ionic strength (e.g., <0.7 M NaCl; see Sambrook & Russell, 2001; see also Murray & Thompson, 1980). An alternative method for isolating plant gDNA is disclosed in Kotchoni & Gachomo (2009) Mol Biol Rep 36:1633-1636. This multi-step method involves grinding plant tissue, incubating the same in a mixture of sodium dodecyl sulfate (SDS) and sodium chloride, spinning down insoluble aggregates, transferring the nucleic acid-containing supernatant to a new vessel, isopropanol precipitation of nucleic acids, re-spinning down the precipitated nucleic acids, performing an ethanol wash, spinning down the washed nucleic acids yet again, drying the nucleic acids, and dissolving the same in a buffer of choice. Some methods for isolating plant gDNA are disclosed in Dilworth & Frey (2000) Plant Molecular Biology Reporter 18:61-64. This method employs Proteinase K and a detergent (e.g., polysorbate 20).

A target species is what the processed botanical material should contain, whereas a non-target species is an adulterant to the processed botanical material. The processed botanical material may or may not contain a non-target species. The processed botanical material may contain one or more non-target species.

Species specific amplification is usually achieved through primers with different hybridization efficiency between target and non-target species. However, the differences between target species and its close relatives may only differ by a single nucleotide residue across all well-characterized barcode regions. In these scenarios, the amplification refractory mutation system (ARMS)-PCR, also known as allele-specific PCR, becomes a good choice. Due to the differences in elongation efficiency of Taq DNA polymerase on 3′-end matched and mismatches primers, in ARMS—PCR, species-specific amplification is achieved when the 3′-end of the primers perfectly complement the base at the template (Newton et al., 1989). To analyze more than one allele in a single PCR assay, Ye et al combined tetra-primer system with ARMS-PCR to form the tetra-primer ARMS-PCR (Ye, Dhillon, Ke, Collins, & Day, 2001). It combines two inner SNP-specific primers and two outer primers in a single reaction and enables simultaneous amplification of the target and alternative alleles plus a DNA quality control. Since the development of tetra-primer ARMS-PCR, it has been employed as a simple and economical tool to rapidly detect known single-nucleotide polymorphism (SNP) in in many areas of study such as pharmacogenetics, genetic disorders, genotyping, and microbiology (Q. Chen et al., 2007; Zabala et al., 2017), but its application in botanical identification and differentiation is rarely reported. Tetra-primer ARMS—PCR can discriminate one species from others by a well characterized SNP. Therefore, it is good for close species identification and differentiation, when regions contain insertion/deletion or multiple mismatches for primer hybridization are not available. Furthermore, Tetra-primer ARMS—PCR features a multiplex PCR system. If present in the sample, the variant allele, characteristic of other species (e.g. adulterant), can also be amplified simultaneously with control from the same region to provide additional information for quality evaluation.

In some embodiments, there are more than one PCR amplicon amplified from the target species. In some embodiments, there are more than one PCR amplicon amplified from the non-target species. The PCR amplicons can be identified by their band sizes based on electrophoresis. The pattern formed from various PCR amplicons from the target species and the pattern from the non-target species are different so that one can tell whether the processed botanical material contains the target species and/or non-target species based on the pattern. In some embodiments, the PCR amplicons are identified by DNA sequencing.

Due to the nature of Taq DNA polymerase based refractory amplification, low amplification cycle number (25-30) is ideal to maintain the specificity of the assay and this assay works best in fresh or lightly processed botanical materials. However, DNA becomes degraded in materials that processed at a higher level. As a result, high amplification cycles (35-40) are required to detect those low-quality DNA. In reactions with high amplification cycle number, tetra-primer ARMS-PCR start to lose its specificity due to the eventual amplification of less favorable allele and sensitivity due to the degradation of the diagnostic inner fragment by the 5′->3′ exonuclease activity in Taq DNA polymerase. This side effect does not only cause false positive and false negative when evaluating botanical materials, but also create different analytical condition requirements for botanical materials at different processing stages. These technical difficulties prevent its wide application in routine botanical quality control.

To solve some of the issues identified above, in some embodiments of this invention, the tetra-primer ARMS-PCR includes a pair of inner primers and a pair of outer primers, and wherein one or both inner primers have a 5′ end random nucleic acid modification and/or a 3′ end phosphorothioate bond modification.

In some embodiments, random nucleotides are added to the 5′ terminus of each inner primer. For example, inner forward primer 3 (Inner F3) has a sequence of NGTCAATACCGGCAACAATGAAATTTT (SEQ ID NO: 7), where N is random nucleotide combination and N can be any nucleotides of A, T, C, and G. More particularly, N represents a combination of A, C, G, and T mixed at roughly equal molar ratio, such as a mixture of AGTCGACGGATTTTCCTCTTAC TAT (SEQ ID NO: 37), CGTCGACGGATTTTCCTCTTACTAT (SEQ ID NO: 38), GGTCGACGGATTTTCCTCTTACTAT (SEQ ID NO: 39), and TGTCGACGGATTTTCCTCTTACTAT (SEQ ID NO: 40) mixed at a roughly equal molar ratio.

Thus, whenever N is used in any of the sequences herein, it designates the combination of A, C, G, and T mixed at a roughly equal molar ratio of the respective sequences. Similarly, whenever V is used in any of the sequences herein, it designates the combination of A, C, and G mixed at roughly equal molar ratio in the respective sequences. Whenever B is used in any of the sequences herein, it designates the combination of C, G, and T mixed at roughly equal molar ratio in the respective sequences. Whenever D is used in any of the sequences herein, it designates the combination of A, T, and G mixed at roughly equal molar ratio in the respective sequences. Whenever H is used in any of the sequences herein, it designates the combination of A, C, and T mixed at roughly equal molar ratio in the respective sequences.

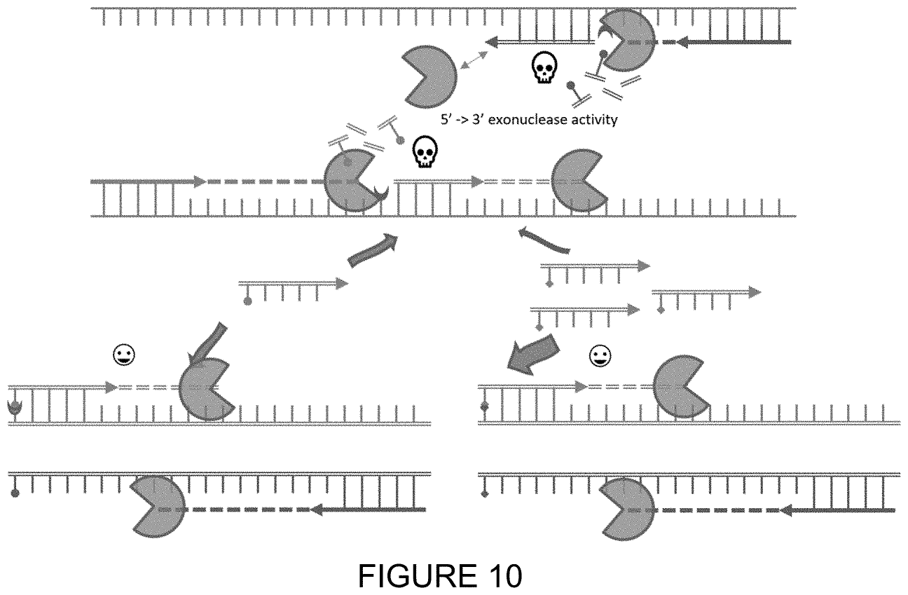

Theoretically, in tetra-primer ARMS-PCR, the inner primer could bind to two types of templates and lead to different fates as illustrated in FIG. 10. When the inner primer binds to a longer template that also contains complement sequence for the outer primer, it has high chance to be hydrolyzed by upstream Taq DNA polymerase while extending from outer primer. In contrast, the inner primer and its daughter strand will not be hydrolyzed when it binds to shorter template (mainly synthesized in the amplification process) that has no upstream binding site for outer primer. Unexpectedly, it was found that using inner primer with a 5′ terminus random nucleotide creates a significant portion of artificial short templates every time inner primers extend by Taq DNA polymerase. As a result, inner primers with 5′ mismatch to wild type sequence shall have better hybridization affinity to artificial short templates than its competitor: the long wild type sequence. In high PCR cycles, when the availability of inner primers is limiting factor, a slightly higher percentage of inner primers and fragments could be saved in each cycle from Taq's 5′−>3′ exonuclease activity, so the dramatic decrease of inner fragment molecules could be delayed (FIG. 10).

The terms “5′ ends” and “3′ ends” or equivalents thereof have their ordinary meaning as understood in light of the specification, and refer to the termini of oligonucleotides because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “complementary” or “complementarity” have their ordinary meaning as understood in light of the specification and are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

In some embodiments, phosphorothioate bonds modification are added to the 3′ terminus of each inner primer.

In some embodiments, each inner primer has at least a 5′ end random nucleic acid modification and at least a 3′ end phosphorothioate bond modification.

In some embodiments, the one or both inner primers have 1-9 3′ end phosphorothioate bonds modification.

In some embodiments, the one or both inner primers have 4 consecutive 3′ end phosphorothioate bonds modification.

In some embodiments, the inner and outer primer ratio is 1:1, 2:1, 3:1, 4:1, 5:1, 6:1, 7:1, 8:1, 9:1, 10:1, 11:1, 12:1, 13:1, 14:1, or 15:1.

In some embodiments, the inner forward primer includes a sequence as set forth in SEQ ID NO: 12 and the inner reverse primer includes a sequence as set forth in SEQ ID NO: 13.

In some embodiments, the inner forward primer includes a sequence as set forth in SEQ ID NO: 31 or 33 and the inner reverse primer includes a sequence as set forth in SEQ ID NO: 32 or 34.

Some embodiments relate to a multiplex PCR system for identifying processed botanical material and optionally detecting adulterant in the material, wherein the processed botanical material comprises a target species and an optional closely related non-target species. The system comprises: 1) an inner forward primer and an inner reverse primer, wherein the 3′ terminus of the inner forward primer form a perfect match with a sequence specific to the target species and the 3′ terminus of the inner reverse primer form a perfect match with a sequence specific to the non-target species, or vice versa, wherein the sequence specific to the target species and the sequence specific to the non-target species differ by a single base or small deletions; and 2) an outer primer pair consisting of an outer forward primer and an outer reverse primer.

In some embodiments, the target species and the non-target species are closely related so that a specific DNA region in the target species and the corresponding region in the non-target species differ only by a single base or a few bases.

In some embodiments, the inner forward and/or reverse primers have a 5′ end random nucleic acid modification, a 3′ end phosphorothioate bonds modification, or both.

In some embodiments, the inner forward and/or reverse primers have 1-9 3′ end phosphorothioate bonds modification.

In some embodiments, the inner forward and/or reverse primers have 4 consecutive 3′ end phosphorothioate bonds modification.

In some embodiments, the multiplex PCR system further comprises a Taq DNA polymerase, or another DNA polymerase that lacks 3′−>5′ exonuclease activity.

In some embodiments, the inner and outer primer ratio is 1:1, 2:1, 3:1, 4:1, 5:1, 6:1, 7:1, 8:1, 9:1, 10:1, 11:1, 12:1, 13:1, 14:1, or 15:1.

In some embodiments, the sample is a market ginseng root material.

In some embodiments, the target species is P. ginseng, and the non-target species is P. quinquefolius, P. notoginseng, P. japonicus, or E. senticosus.

Some embodiments relate to a method using a multiplex PCR system for identifying and differentiating a target species from a closely related non-target species in a sample. The method comprises: 1) identifying a specific DNA region that differs by a single base or small deletions between the target species and the non-target species; 2) designing an inner forward primer, an inner reverse primer, an outer forward primer, and an outer reverse primer, wherein the 3′ terminus of the inner forward primer form a perfect match with a sequence specific to the target species and the 3′ terminus of the inner reverse primer form a perfect match with a sequence specific to the non-target species, or vice versa, wherein the sequence specific to the target species and the sequence specific to the non-target species differ by a single base or small deletions; 3) providing a sample suspected of containing both a target species and a closely related non-target species; and 4) conducting PCR reaction using the sample to identify the target species and/or non-target species in the sample.

Some embodiments relate to a multiplex PCR kit for identifying and differentiating a target species from a closely related non-target species in a sample, said kit comprises: 1) an inner forward primer and an inner reverse primer, wherein the 3′ terminus of the inner forward primer form a perfect match with a sequence specific to the target species and the 3′ terminus of the inner reverse primer form a perfect match with a sequence specific to the non-target species, or vice versa, wherein the sequence specific to the target species and the sequence specific to the non-target species differ by a single base or small deletions; 2) an outer primer pair consisting of an outer forward primer and an outer reverse primer; and 3) a Taq DNA polymerase, or another DNA polymerase that lacks 3′−>5′ exonuclease activity.

In some embodiments, the inner forward and/or reverse primers have a 5′ end random nucleic acid modification and/or a 3′ end phosphorothioate bond modification.

In some embodiments of this invention, using P. ginseng identification as an example, a tetra-primer ARMS-PCR method was developed to identify and differentiate P. ginseng from other species in Panax spp. and botanicals that have “ginseng” in their common name by a characteristic SNP observed only in P. ginseng genome. In some embodiments, the method's application scope can be expanded to cover popular industry material types at different process stages and unify test conditions for increased practicality, multiple technical improvements were made in the current optimized tetra-primer ARMS-PCR assay. In some embodiments, the novel features of the tetra-primer ARMS-PCR method include 1) a 5′ end random nucleic acid modification to rescue the degradation of the inner fragment caused by Taq DNA polymerase and 2) a 3′ end phosphorothioate bonds modification to further reduce the mismatch allele elongation efficiency. In some embodiments, the tetra-primer ARMS-PCR test condition was validated using market ginseng root materials at different processing stages and in different mixed status.

In some embodiments, the amplification refractory mutation system-polymerase chain reaction (ARMS-PCR) is used in a method for detecting any mutation involving single base changes or small deletions. The tetra-primer ARMS-PCR uses 2 sets or 4 primers.

### EXAMPLES

Embodiments of the present invention are further defined in the following Examples. It should be understood that these Examples are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims. The disclosure of each reference set forth herein is incorporated herein by reference in its entirety.

### Example 1

This example describes an oligo modification strategy to unify tetra-primer ARMS-PCR conditions for P. ginseng identification and differentiation at difference processing stages. Panax ginseng could be adulterated with Panax quinquefolius, or vice versa, depends on the price fluctuation of specific parts and the end-user market country. Using unclaimed ginseng species is not only a regulatory issue, but also compromises consumer confidence and leads to unexpected efficacy even safety concerns. Following the same example, although both P. ginseng and P. quinquefolius contains ginsenosides as their bioactive compound and recognized as adaptogens (herbs to restore equilibrium and resist to adverse factors) in general, they are intended for different therapeutic outcomes. In TCM, P. ginseng is stimulating and invigorates “Yang”, whereas P. quinquefolius is calming and nourishing “Yin”. Modern research also suggests the contents of individual components are more important than the total ginsenosides (Chen, Chiou, & Zhang, 2008). For example, ginsenoside Rg1 and Rb 1 enhance Central Nervous System (CNS) and modulate angiogenesis activities, but the effect of the latter is weaker, sometimes even inhibitory (Chang, Huang, Tien, & Wang, 2008; Sengupta et al., 2004). Typically, P. ginseng root has a higher Rg1/Rb1 content ratio to support its stimulating effects, while P. quinquefolius root's low Rg1/Rb1 content ratio explains its calming effects.

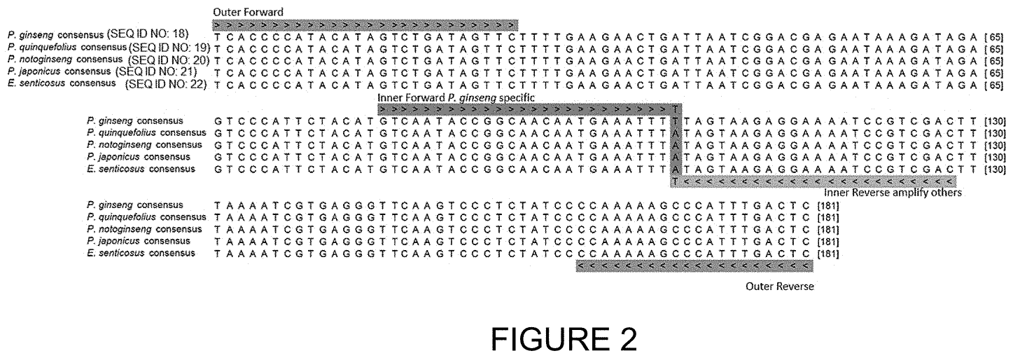

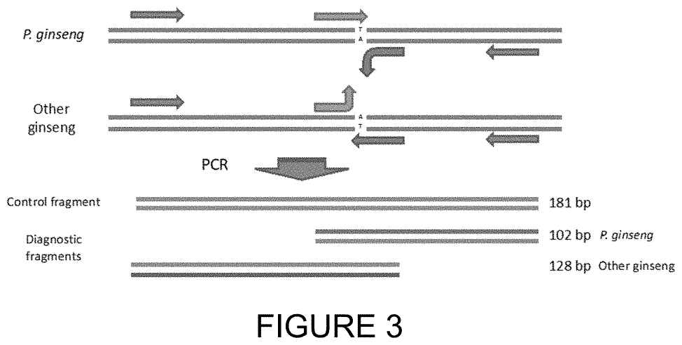

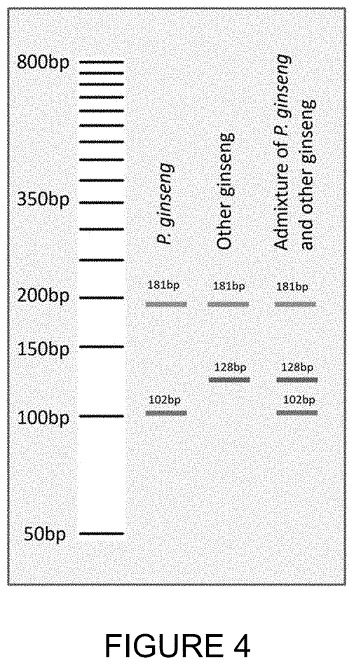

To identify SNP candidates that are unique to P. ginseng, common barcode regions of P. ginseng and its close species were downloaded from GenBank, aligned, and assessed for intra- and intergenic variations. A P. ginseng specific SNP observed within trnL-trnF region was selected for designing tetra-primer ARMS-PCR assay due to its balanced GC content. As shown in FIG. 2, the top strand of P. ginseng consensus sequence exhibits Thymine (T) at the SNP position, while other ginseng species, such as P. quinquefolius, P. notoginseng, P. japonicus, and E. senticosus exhibit adenine (A). Four primers (SEQ ID NOs: 1-4) are designed around the SNP position to give PCR fragments in different lengths for P. ginseng and other species: the outer primer pair amplifies a large 181 bp internal control fragment from all genomic DNA in the test scope, while the inner primers amplify two smaller diagnostic fragments representing two allelic states in P. ginseng and other species. When P. ginseng DNA is present, a perfect match is formed between the P. ginseng specific inner forward primer and DNA template from P. ginseng; paring the inner forward primer with the outer reverse primer resulting in a 102 bp fragment. On the other hand, the inner reverse primer could pair with outer forward primer to generate a 128 bp fragment only when a perfect match is formed between the 3′ terminus of inner reverse primer and template DNA from other ginseng species (FIG. 3). In the case of mismatch, neither inner primer can be efficiently extended at the 3′ terminus alternative allele position by Taq DNA polymerase due to the presence of T:T mismatch, which exhibits maximum destabilization strength among all mismatches (Stephen Little, 1995). Based on the characteristic size of each inner fragment, the identity of P. ginseng and other ginseng can be easily resolved using DNA visualization equipment or agarose gel electrophoresis (FIG. 4).

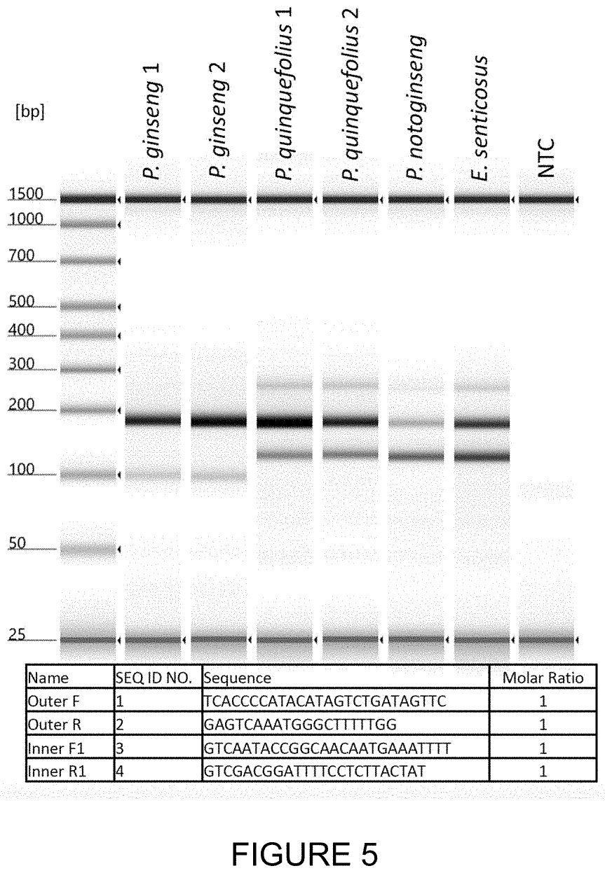

To test its feasibility in P. ginseng identification and differentiation, the current tetra-primer ARMS-PCR was applied to DNA extracted from reference materials of multiple Panax spp. and E. senticosus. As shown in FIG. 5, with 28 cycles of PCR amplification, DNA extracted from P. ginseng reference materials generated a large fragment around 180 bp and a small fragment around 100 bp, while DNA from P. quinquefolius, P. notoginseng, and E. senticosus yielded the same 180 bp large fragment and another distinctive small fragment with size around 128 bp. Although a few non-specific bands were observed in certain species, the region between large and small fragments is relative clean. Based on the size difference of small fragments, P. ginseng reference material can be easily distinguished from reference material of other species in the test scope.

In addition to rapid species differentiation, appropriate control is often required to assess PCR inhibitors and DNA degradation, which are major impediments to successful DNA amplification of plant DNA (Porebski, Bailey, & Baum, 1997; Ruhsam & Hollingsworth, 2018). PCR inhibitors, derived from various plant compounds, such as polysaccharides and certain secondary metabolites in plant tissues, may co-precipitate with DNA to inhibit enzyme activity in PCR amplification. Unfortunately, the Taq DNA polymerase, which is important for tetra-primer ARMS-PCR, is among the most sensitive enzyme to PCR inhibition (Abu Al-Soud & Radstrom, 1998). DNA degradation, particularly in the case of dietary supplements, also prevent the labeled botanical ingredients to be reproducibly identified (Arulandhu et al., 2017). Conventionally, endogenous or exogenous DNA is co-amplified besides target region to function as a control to assist the assessment of PCR inhibitors. However, due to factors, such as the control and target resides in different locations of the genome, different PCR amplification efficiency and DNA degradation level between control and target region, a positive amplification of control fragment in end-point PCR does not necessarily mean the amplification of target fragment is not compromised. In some cases, the target region in the inhibited samples is even mistakenly assumed to be degraded. In contrast, the current tetra-primer ARMS-PCR method offers a direct assessment of both PCR inhibitor and DNA degradation. The successful amplification of control fragment by outer primer pair indicates not only the target region is amplifiable but also the template required for diagnostic fragment amplification is intact, therefore the absence of small diagnostic fragment could be confidently interpreted as true negative.

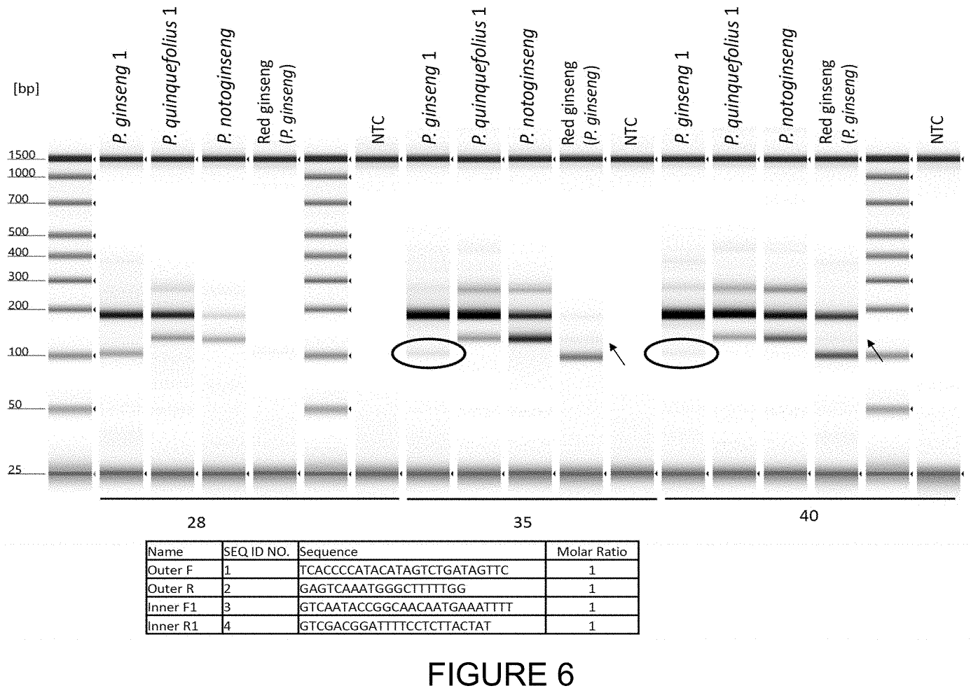

There are numerous P. ginseng products on the market. They are all derived from the same plant species but undergo different processing. As a result, characteristic chemical profiles exist in different P. ginseng products, so does the chemical analytical methods and acceptance criteria used to identify them (Lee et al., 2015). During processing, genomic information used by DNA-based analytical methods is not altered. However, drying, steaming, and extraction significantly compromise botanical DNA in terms of quality and quantity, therefore frequently resulting in false negative due to the failure of amplifying the characteristic fragment (Ragupathy et al., 2019). To make the current tetra-primer ARMS-PCR applicable in highly processed P. ginseng products, the size of the large control amplicon was designed to be less than 200 bp based on previous knowledge on fragment size and higher PCR cycle numbers were used to compensate the reduction in template quantity (Lu et al., 2018). For processed P. ginseng products, P. ginseng extract and red ginseng are the two major types. P. ginseng extract was not included in the optimization study, since ginseng extract is manufactured using bulk root materials, which may not necessarily contain pure P. ginseng from a genomic point of view. Previously authenticated single root red ginseng product was included in current evaluation to represent highly processed ginseng products. FIG. 6 shows the PCR amplicons profile generated using DNA extracted from botanical reference material and red ginseng at 28, 35 and 40 cycles, respectively. Conditions with low PCR cycle number (28 cycles) works for botanical reference material as expected. However, the same condition only yielded a faint small inner fragment, but not the large control fragment for the red ginseng sample. With the increments of PCR cycle number (35 and 40 cycles), both small and large fragments became visible for the red ginseng sample, suggesting the current assay was able to retrieve degraded DNA from highly processed P. ginseng products. However, the other 130 bp inner band, indicating the presence of other ginseng species was also visible and hard to be ignored (arrows). Since the red ginseng DNA was extracted from a single root, the presence of both diagnostic bands indicates a specificity issue of the current tetra-primer ARMS-PCR used at high cycle number. In addition, although objective and rapid species identification could be achieved in reference materials DNA with low PCR cycle number, high PCR cycle numbers that required for processed material did lead to lower sensitivity featured by the reduction of small diagnostic fragment intensity, especially for the target species P. ginseng (circles).

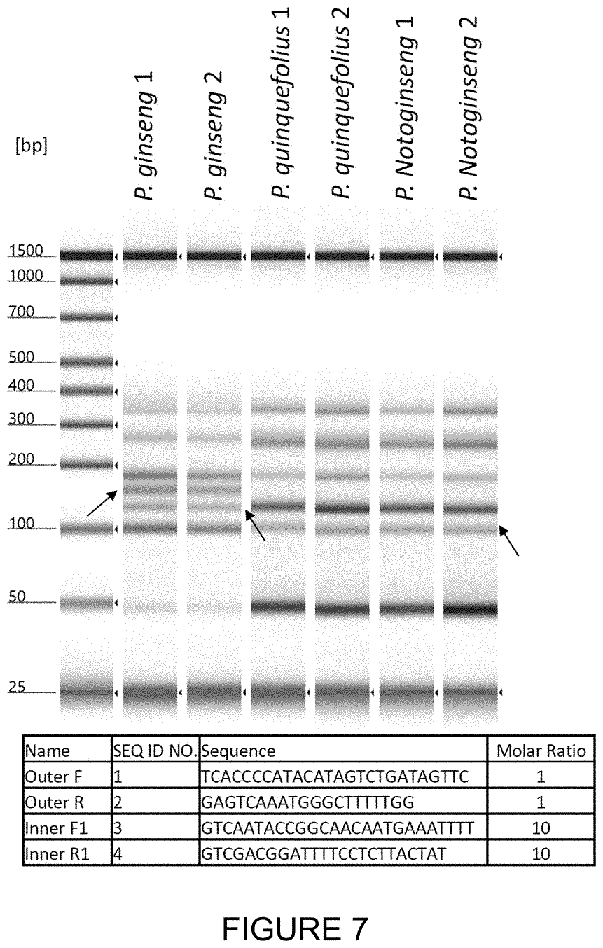

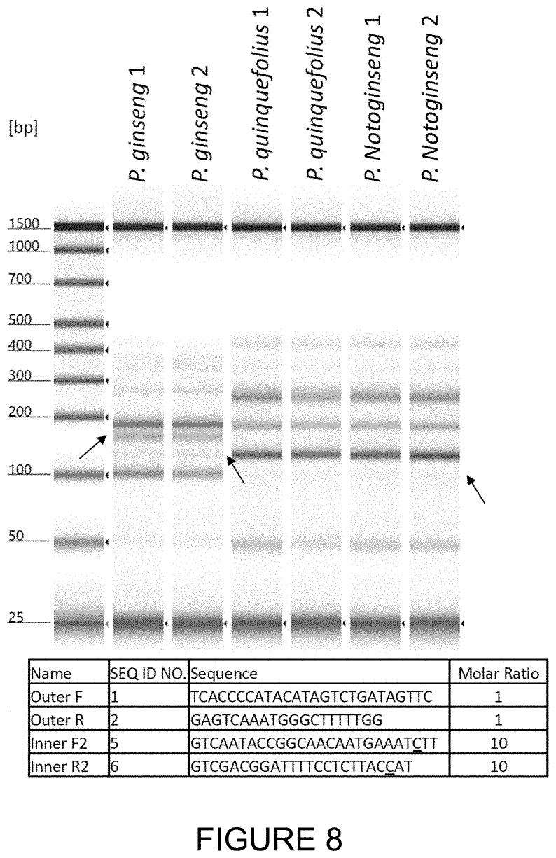

To improve both the sensitivity and specificity of current P. ginseng assay, 10:1 inner (SEQ ID NOs: 3-4) vs. outer (SEQ ID NOs: 1-2) primer ratio and additional deliberate mismatch at -2 position from 3′ terminus of inner primers (SEQ ID NOs: 5-6) was introduced based on original tetra-primer ARMS-PCR design website. The performance of new assay was evaluated using additional Panax spp. materials at 40 PCR cycles. As illustrated in FIG. 7, simply increasing inner vs. outer primer ratio (SEQ ID NOs: 3-4 vs SEQ ID NOs: 1-2 at a 10:1 ratio) yielded a well-balanced small and large fragment intensity. However, the selectivity of inner primer was also lost due to excessive amount of inner primers. Adding a deliberate mismatch at -2 position (SEQ ID NOs: 5-6) significantly improves the specific amplification of diagnostic fragment (FIG. 8). However, low level amplification of the other diagnostic fragment was not completely eliminated (arrows in FIG. 8). Furthermore, non-specific amplifications were also observed in all P. ginseng samples (left most arrows in FIGS. 7 and 8). Current data suggests the classical strategy did not achieve the desired sensitivity and specificity at the same time and additional optimization is required.

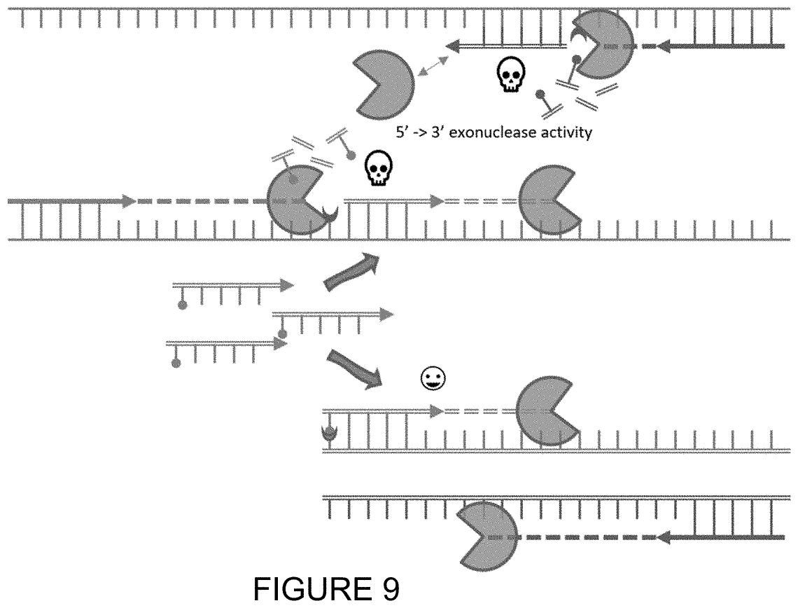

The 3′ terminus mismatch prevents DNA polymerase from extending the mismatched primer. The Taq DNA polymerase is important to ARMS-PCR due to its lack of 3′−>5′ exonuclease activity, so it cannot excise the 3′ terminus mismatched base like other proofreading polymerases (Eom, Wang, & Steitz, 1996). However, Taq DNA polymerase also possesses 5′−>3′ exonuclease activity (TaqMan activity), which can hydrolyze the downstream non-template inner fragment primer and its daughter strand while extending from the outer primer binds to the same DNA template (FIG. 9) (Li, Mitaxov, & Waksman, 1999). Various strategies has been tested to reduce Taq DNA polymerase's undesired activity on bound inner primer, including antisense oligonucleotides to block outer primers, using chemically modified inner primers, and replacing Taq with Q5 DNA polymerase that lacks 5′ −>3′ exonuclease activity (Shatleh-Rantisi, Tamimi, & Ashhab, 2020). Although Q5 DNA polymerase achieves best result, its 3′−>5′ exonuclease activity prevent it to be used in ARMS-PCR.

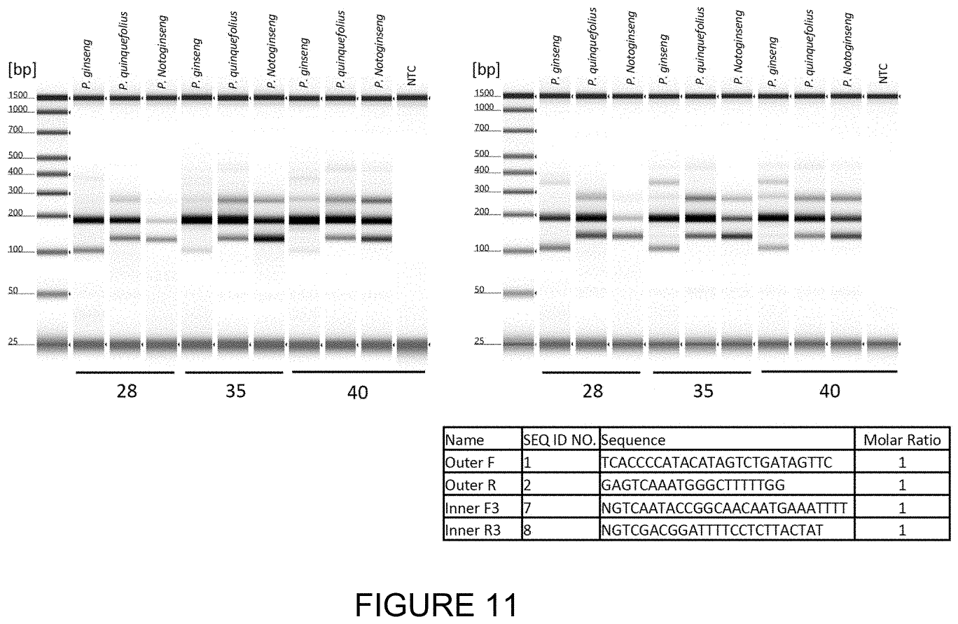

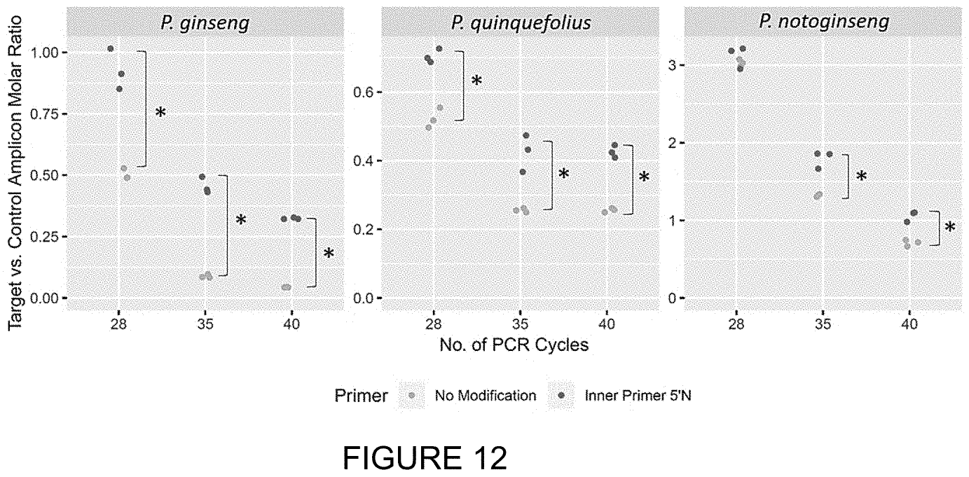

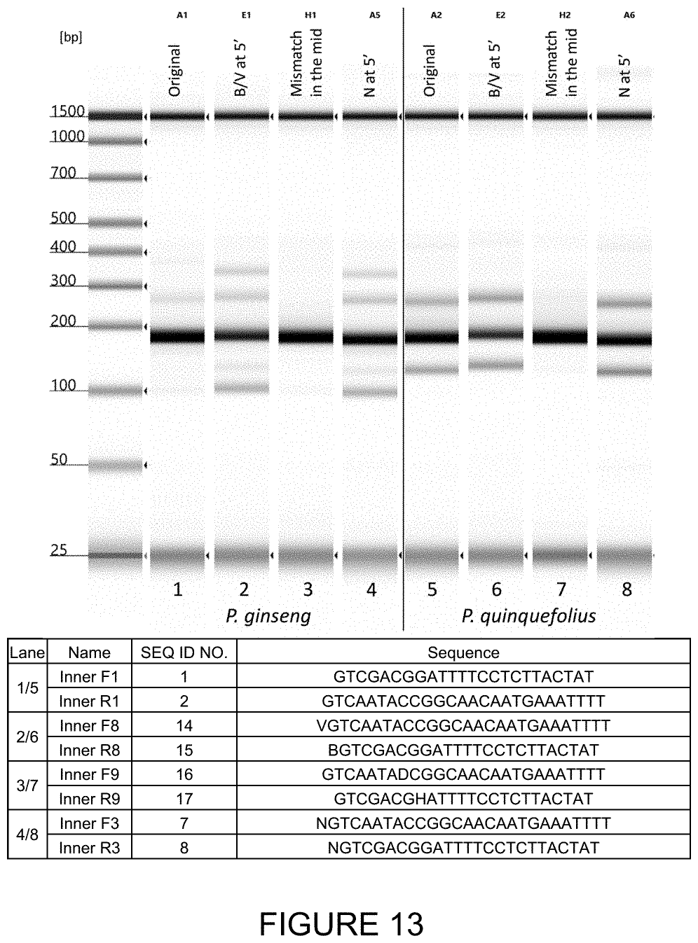

Previous attempts showed that supplying the tetra-primer ARMS-PCR reaction with excessive amount of inner primers may result in non-specific amplification of second inner fragment that represents the other allelic state at high PCR cycles. An alternative strategy that adding a random nucleotide to the 5′ terminus of each inner primer was tested for its effects on improving assay sensitivity. Theoretically, in tetra-primer ARMS-PCR, the inner primer could bind to two types of templates and lead to different fates as illustrated in FIG. 10. When the inner primer binds to a longer template that also contains complement sequence for the outer primer, it has high chance to be hydrolyzed by upstream Taq DNA polymerase while extending from outer primer. In contrast, the inner primer and its daughter strand will not be hydrolyzed when it binds to shorter template (mainly synthesized in the amplification process) that has no upstream binding site for outer primer. Unexpectedly, it was found that using inner primer with a 5′ terminus random nucleotide creates a significant portion of artificial short templates every time inner primers extend by Taq DNA polymerase. As a result, inner primers with 5′ mismatch to wild type sequence shall have better hybridization affinity to artificial short templates than its competitor: the long wild type sequence. In high PCR cycles, when the availability of inner primers is limiting factor, a slightly higher percentage of inner primers and fragments could be saved in each cycle from Taq's 5′−>3′ exonuclease activity, so the dramatic decrease of inner fragment molecules could be delayed (FIG. 10). To test this hypothesis, inner primer set with a 5′ terminus random nucleotide (SEQ ID NOs: 7-8) was compared to regular inner primer on all DNA from Panax spp. FIG. 11 demonstrated that replacing regular inner primer (right panel) with inner primer set with a 5′ terminus random nucleotide (left panel) boosted the intensity of inner fragment across various PCR cycles. To further quantify the improvement of sensitivity, the molar ratio between the desired inner and control fragments was obtained from TapeStation analysis software in triplicate. All tested ginseng species DNA showed higher inner vs. outer fragment molar ratio when using inner primer with 5′ terminus random nucleotides at 35 and 40 cycles (p <0.05, student t-test), suggesting adding 5′ terminus random nucleotides on inner primers can significantly improve assay sensitivity (FIG. 12). In addition to random nucleotides on the 5′ terminus, additional variations of mismatches were also tested (FIG. 13) (SEQ ID NOs: 9-12). However, adding random nucleotides on the 5′ terminus of inner primer is the most efficient and straight forward solution.

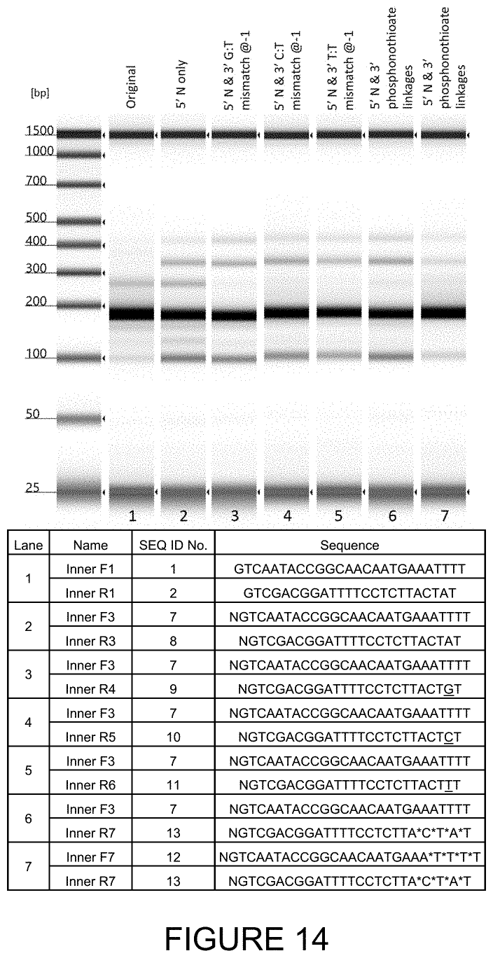

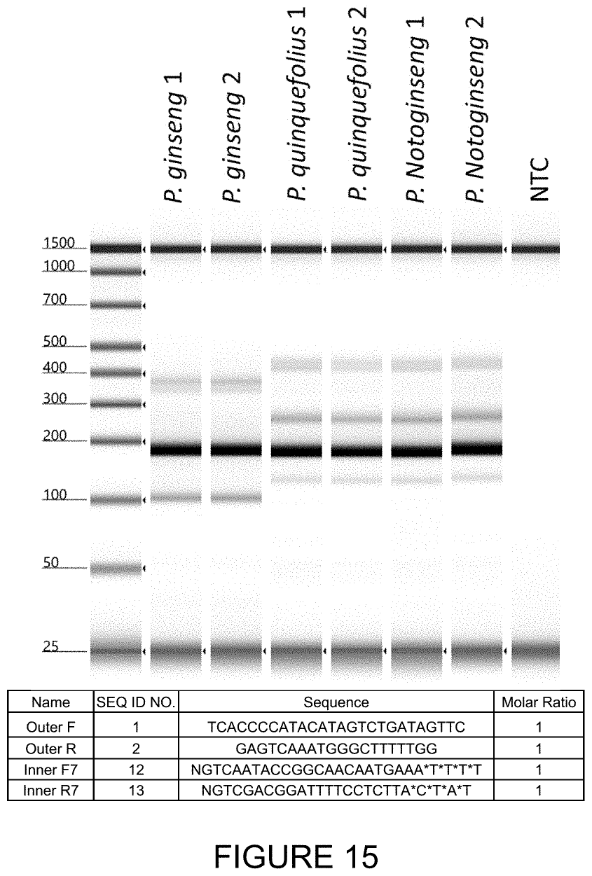

The specificity of tetra-primer ARMS-PCR is mainly depends on the reduced elongation efficiency of Taq DNA polymerase at template-primer 3′-terminus with mismatched base pairs (Huang, Arnheim, & Goodman, 1992). Ye et al. suggested that additional deliberate mismatch at -2 position from 3′ terminus could further improves specificity (Ye et al., 2001). However, in the current assay, two mismatches at both 3′ terminus and −2 position from 3′ terminus were not enough to inhibit the amplification of the second inner fragment at 40 PCR cycles. To further improve the specificity of the assay, two strategies have been tested: 1) introduce deliberate mismatch at penultimate base (−1) instead of −2 position to deliver a more direct effect on decreasing elongation efficiency (S Little, 2001); 2) replacing the 3′ end regular phosphodiester internucleotide linkages with phosphorothioate linkages. Phosphorothioate linkages not only make the oligo more resistant to nuclease, but also render the oligo more rigid, therefore improves specificity (Heissl, Arbeithuber, & Tiemann-Boege, 2017). As shown in FIG. 14, while a mismatch at the penultimate base did reduce the amplification of the second inner fragment (for example, by replacing SEQ ID NO: 8 with SEQ ID NO: 13, 14, or 15), using 4 consecutive phosphorothioate linkages at 3′ terminus (for example, by replacing SEQ ID NOs: 7-8 with SEQ ID NOs: 16-17) completely blocked the amplification of the other inner fragment frequently observed in P. ginseng samples. Apply primers with 3′ phosphorothioate linkages to the same set of reference used in assess classical strategy confirmed the improved specificity (FIG. 15).

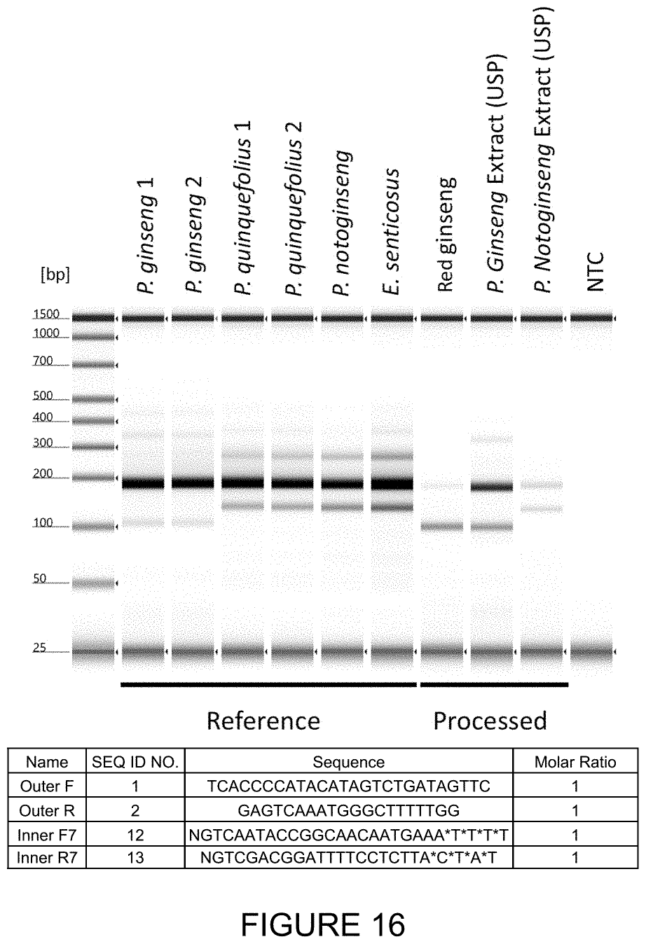

To achieve high sensitivity and specificity of in current P. ginseng tetra-primer ARMS-PCR, both 5′ end random nucleotide and 3′ terminus phosphorothioate linkage modifications were incorporated into inner primers. The result indicates that, with modified inner primers, a universal assay condition can be applied with acceptable sensitivity and specificity to DNA extracted from ginseng products at different processing stages, ranging from lightly processed botanical reference materials, steamed roots, even extracts (FIG. 16).

All oligos were purchased from Integrated DNA Technologies (Coralville, Iowa, USA).

Outer forward primer:
(SEQ ID NO: 1)
5′-TCACCCCATACATAGTCTGATAGTTC-3′,
Outer reverse primer:
(SEQ ID NO: 2)
5′-GAGTCAAATGGGCTTTTTGG-3′,
Inner forward primer (regular, P. ginseng specific):
(SEQ ID NO: 3)
5′-GTCAATACCGGCAACAATGAAATTTT-3′ (inner F1),
Inner reverse primer (regular, other ginseng specific):
(SEQ ID NO: 4)
5′-GTCGACGGATTTTCCTCTTACTAT-3′ (inner R1),
Inner forward primer (regular):
(SEQ ID NO: 5)
5′-GTCAATACCGGCAACAATGAAATCTT-3′ (inner F2),
Inner reverse primer (regular):
(SEQ ID NO: 6)
5′-GTCGACGGATTTTCCTCTTACCAT-3′ (inner R2),
Inner forward primer (5′ modification):
(SEQ ID NO: 7)
5′-NGTCAATACCGGCAACAATGAAATTTT-3′ (inner F3),
Inner reverse primer (5′ modification):
(SEQ ID NO: 8)
5′-NGTCGACGGATTTTCCTCTTACTAT-3′ (inner R3),
Inner forward primer (5′ modification):
(SEQ ID NO: 9)
5′-VGTCAATACCGGCAACAATGAAATTTT-3′ (inner F8),
Inner reverse primer (5′ modification):
(SEQ ID NO: 10)
5′-BGTCGACGGATTTTCCTCTTACTAT-3′ (inner R8),
Inner forward primer (middle modification):
(SEQ ID NO: 11)
5′-GTCAATADCGGCAACAATGAAATTTT-3′ (inner F9),
Inner reverse primer (middle modification):
(SEQ ID NO: 12)
5′-GTCGACGHATTTTCCTCTTACTAT-3′ (inner R9),
Inner reverse primer (5′ and 3′ modification):
(SEQ ID NO: 13)
5′-NGTCGACGHATTTTCCTCTTACTGT-3′ (inner R4),
Inner reverse primer (5′ and 3′ modification):
(SEQ ID NO: 14)
5′-NGTCGACGHATTTTCCTCTTACTCT-3′ (inner R5),
Inner reverse primer (5′ and 3′ modification):
(SEQ ID NO: 15)
5′-GTCGACGHATTTTCCTCTTACTTT-3′ (inner R6),
Inner forward primer:
(SEQ ID NO: 16)
5′-NGTCAATACCGGCAACAATGAAA*T*T*T*T-3′ (inner F7),
Inner reverse primer:
(SEQ ID NO: 17)
5′-NGTCGACGGATTTTCCTCTTA*C*T*A*T-3′ (inner R7).

(Note: N, random nucleotides; *, phosphorothioate bond)

The tetra-primer ARMS-PCR assay was performed in a 20 μL PCR mix, containing 10 μL AmpliTaq Gold™ 360 Master Mix, 2 μL primer mix (with 5 μM each primer), 2 μL gDNA extracted from ginseng materials, and 6 μL nuclease-free water.

The following thermal profile was used: (1) an initial step of 3 min at 95° C. and (2) 40 cycles of 30 s at 95° C., 30 s at 60° C., and 30 s at 72° C. and (3) final extension 3 min at 72° C.

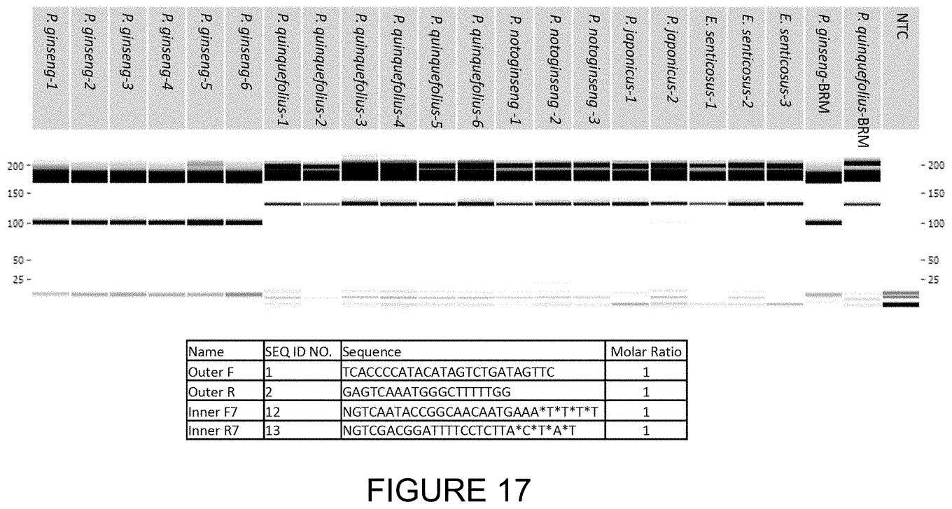

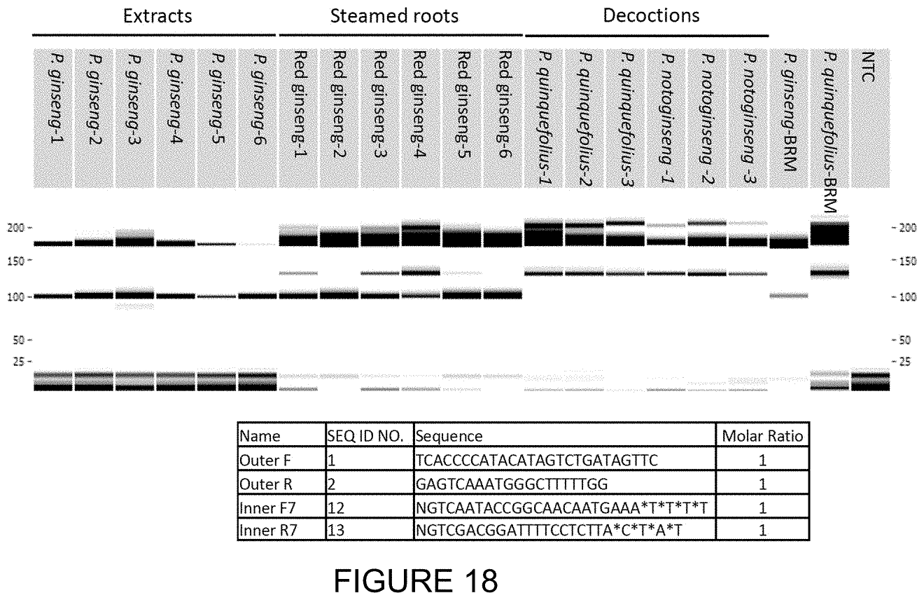

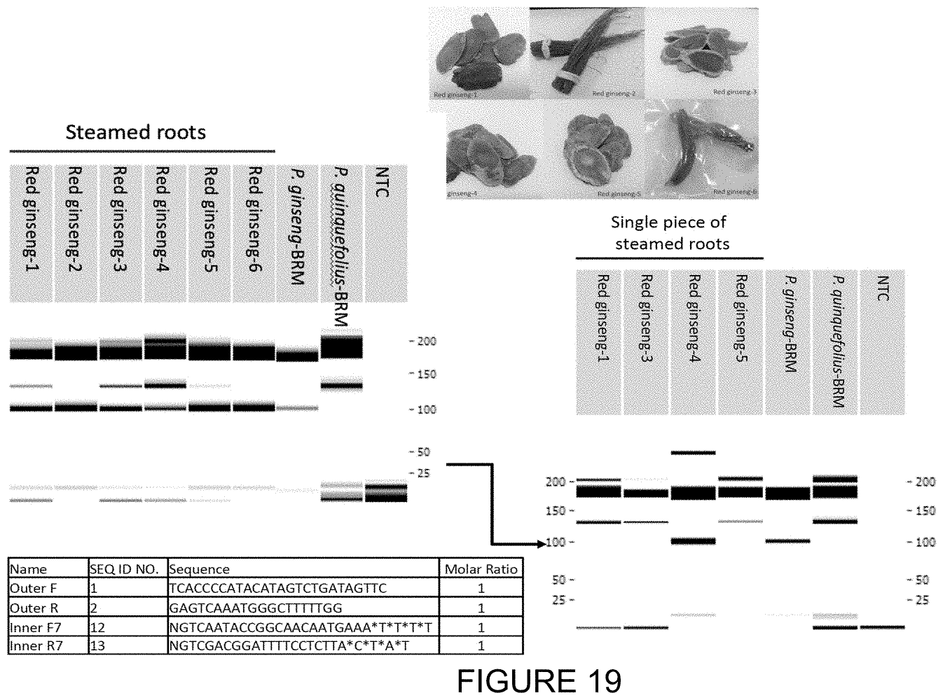

To evaluate the applicability of unified P. ginseng tetra-primer ARMS-PCR assay condition, market samples from multiple sources were tested under the same condition listed above. For unprocessed or lightly processed root samples (drying), current assay was able to identify 6 lots of P. ginseng samples as P. ginseng, and gave different pattern for 6 lots of P. quinquefolius, 6 lots of P. notoginseng, 2 lots of P. japonicus, and 3 lots of E. senticosus (FIG. 17). For highly processed samples (high temperature treatment or extraction), current assay was able to identify 6 lots of P. ginseng extract samples as P. ginseng, and gave different pattern for 3 lots of P. quinquefolius decoction, and 3 lots of P. notoginseng decoction (FIG. 18). Testing on Red ginseng samples yielded inconsistent result, later examination of the samples reveal they exist in different status (FIG. 19). Two whole root samples yielded P. ginseng pattern as expected, while root slices give pattern suggest a mixture of P. ginseng and other ginseng species. Additional DNA extraction was performed using a single slice, pure P. ginseng or other ginseng conclusion was drawn based on the second attempt, suggesting tetra-primer ARMS-PCR assay with modified inner primers is able to deliver specific result and is sensitive to adulteration in mixed states. Additional lots of market ginseng samples and decoctions were also tested at a different laboratory with inner primer modification (Table 1).

TABLE 1
Ginseng and related species authentication by allele-specific PCR
Raw herbal materials (5 g of samples were also boiled
in 100 ml water to prepare decoction)

| | | Authentication results | |
|---|---|---|---|
| T code | Herbal materials | Raw materials | Decoction |

Claimed as P. ginseng

| | | | |
|---|---|---|---|
| T3365 | Whole root raw Asian ginseng | P. ginseng | P. ginseng |
| T3890 | Asian ginseng | P. ginseng | P. ginseng |
| T4130 | White ginseng | P. ginseng | P. ginseng |
| T5254 | Korean ginseng rhizome | P. ginseng | P. ginseng |
| T5255 | Korean ginseng | P. ginseng | P. ginseng |
| T5256 | Korean ginseng | P. ginseng | P. ginseng |

Claimed as P. quinquefolius

| | | | |
|---|---|---|---|
| T5257 | Premium American ginseng | Other ginseng | Other ginseng |
| | slice | | |
| T5258 | American ginseng, size three, | Other ginseng | Other ginseng |
| | two faces | | |
| T5259 | Premium Canadian ginseng | Other ginseng | Other ginseng |
| | slice | | |
| T5260 | American ginseng, jiankou | Other ginseng | Other ginseng |
| T5261 | American ginseng, | Other ginseng | Other ginseng |
| | yuanweixiaozhi | | |
| T2862 | American ginseng cuxu | Other ginseng | Other ginseng |

Claimed as P. notoginseng

| | | | |
|---|---|---|---|
| T2860 | Tienchi | Other ginseng | N.A. |
| T2865 | Tienchi | Other ginseng | N.A. |
| T3592 | Sanqi | Other ginseng | N.A. |

Claimed as Pseudostellaria heterophylla

| | | | |
|---|---|---|---|
| T1748 | Taizishen | Other ginseng | N.A. |
| T3495 | Taizishen | Other ginseng | N.A. |
| T3891 | Taizishen | Other ginseng | N.A. |

Claimed as A. senticosus

| | | | |
|---|---|---|---|
| T4195 | Siberian ginseng | Other ginseng | N.A. |

### Example 2

This example tests the adulteration detection limit of some embodiments.

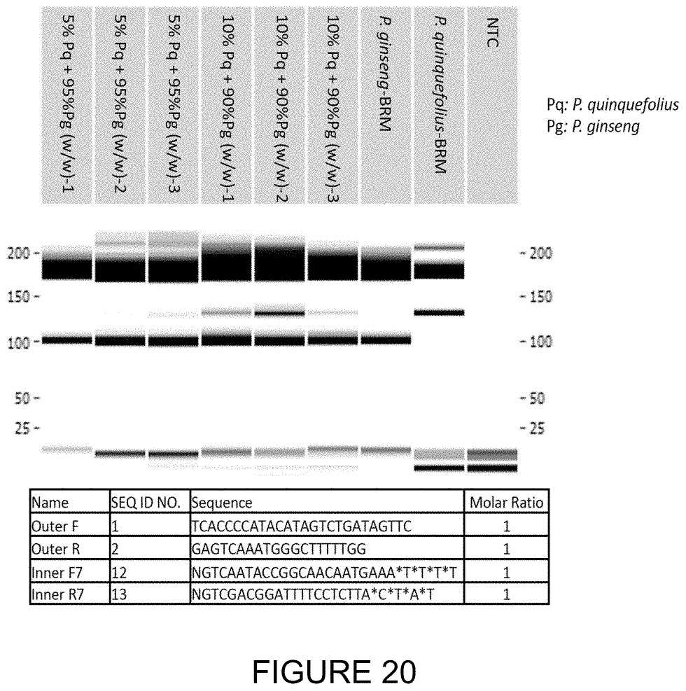

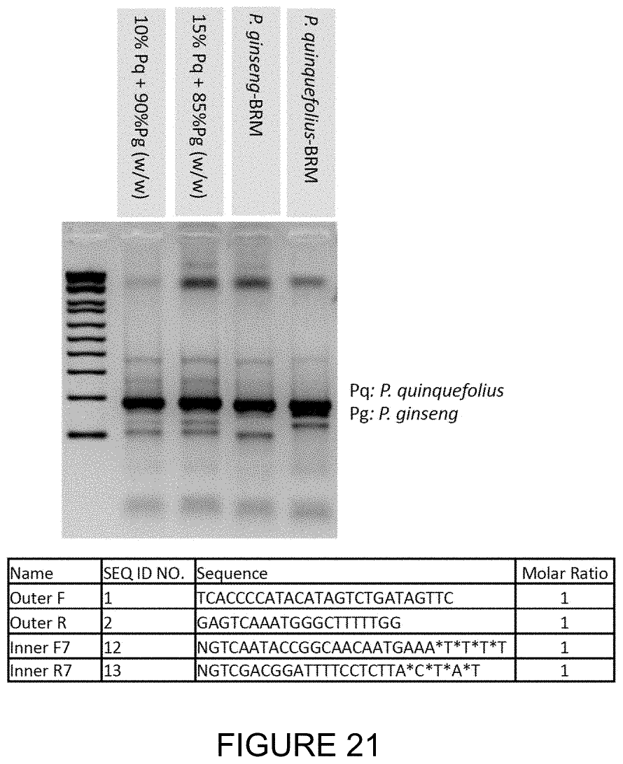

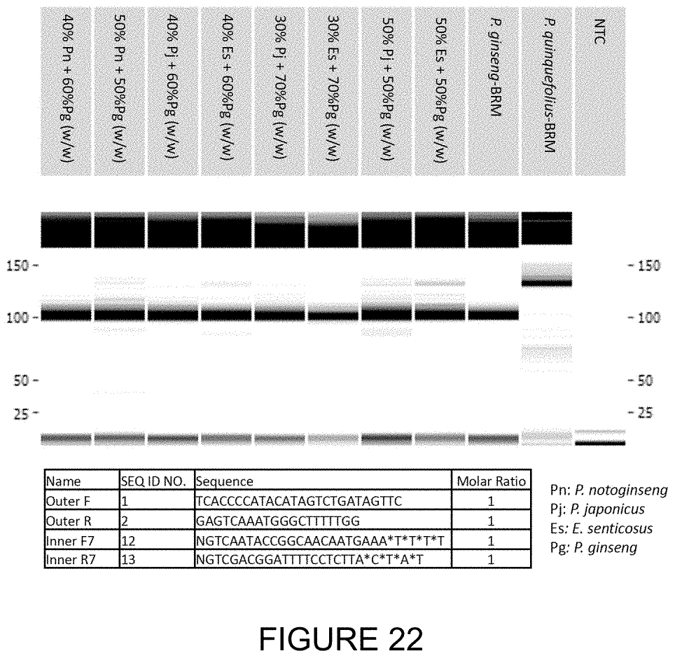

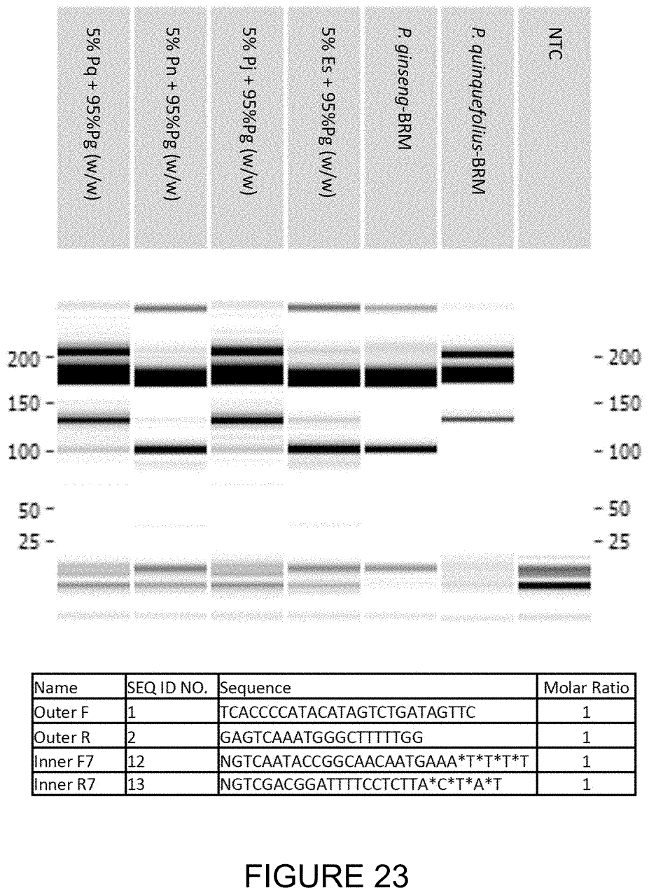

To further test the adulteration detection limit of the modified assay, ginseng materials were fixed using market samples. Since DNA template ratio is the most critical factor that influence test sensitivity, to avoid testing all P. ginseng and other ginseng combinations, one P. ginseng sample has the highest DNA concentration were combined with one of each other species samples that showed lowest DNA concentration in DNA extraction, with the assumption that if adulteration can be detected in this status, then it can also be detected in samples mixed using material have higher DNA concentration at same weight percentage. For unprocessed or lightly processed materials, P. quinquefolius adulteration in P. ginseng can be consistently detected using capillary electrophoresis (FIG. 20, 10%/90% weight/weight) and regular agarose gel electrophoresis (FIG. 21), while detection of P. notoginseng, P. japonicus, and E. senticosus adulteration in P. ginseng were achieved at 50%, 50%, 40% (w/w), likely due to low DNA extraction efficiency in other ginseng root materials (FIG. 22). Further, current assay reaches 5% (w/w) detection sensitivity in mixed materials after they were made into decoction (FIG. 23).

### Example 3

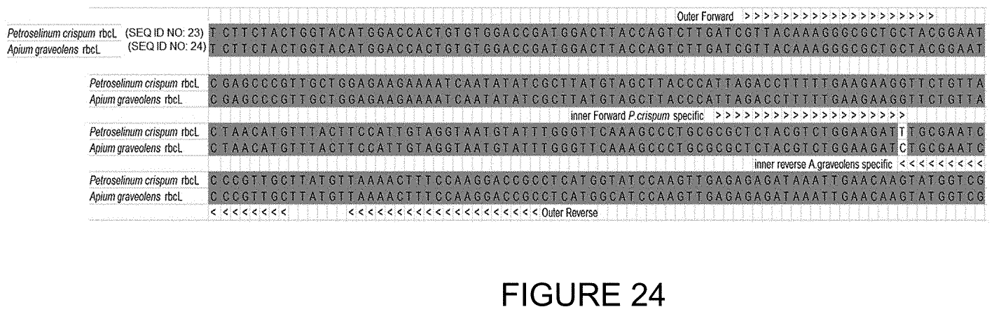

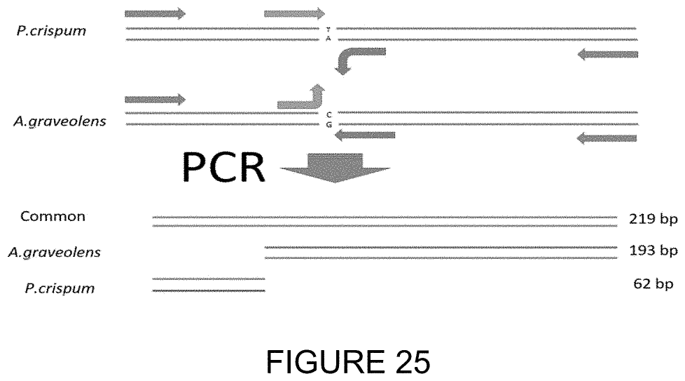

To further demonstrate the application of this invention in the unification of tetra-primer amplification refractory mutation system-polymerase chain reaction (ARMS-PCR) conditions for botanical materials at difference processing stages other than Panax spp., ARMS-PCR were also designed for parsley (Petroselinum crispum) and celery (Apium graveolens) differentiation. P. crispum and A. graveolens leaf flakes share many morphological and chemical characteristics. However, they can be easily differentiated by genomic methods (Quan et al., 2020). To identify SNP candidates that are unique to P. crispum, common barcode regions of P. crispum and A. graveolens were downloaded from GenBank, aligned, and assessed for intra- and inter- genic variations. A P. crispum specific SNP observed within rbcL region was selected for designing tetra-primer ARMS-PCR assay. As shown in FIG. 24, the top strand of P. crispum consensus sequence exhibits Thymine (T) at the SNP position, whereas A. graveolens exhibits cytosine (C). Four primers (SEQ ID NOs: 27-30) are designed around the SNP position to give PCR fragments in different lengths for P. crispum and A. graveolens. The outer primer pair amplifies a large 219 bp internal control fragment from all genomic DNA in the test scope, whereas the inner primers amplify two smaller diagnostic fragments representing two allelic states in P. crispum and A. graveolens. When P. crispum DNA is present, a perfect match is formed between the P. crispum specific inner forward primer and DNA template from P. crispum, paring the inner forward primer with the outer reverse primer resulting in a 62 bp fragment. On the other hand, the inner reverse primer could pair with outer forward primer to generate a 193 bp fragment only when a perfect match is formed between the 3′ terminus of inner reverse primer and template DNA from other A. graveolens (FIG. 25). In the case of mismatch, neither inner primer can be efficiently extended at the 3′ terminus alternative allele position by Taq DNA polymerase. Based on the characteristic size of each inner fragment, the identity of P. crispum and A. graveolens can be resolved using DNA visualization equipment or agarose gel electrophoresis.

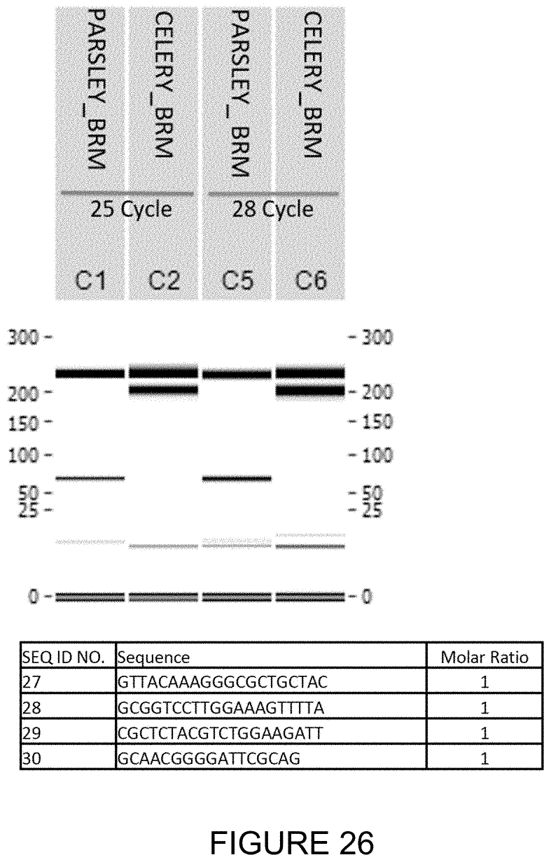

The current design was first tested using low PCR cycles and primers without any modifications. As shown in FIG. 26, with 25 and 28 cycles of PCR amplification, DNA extracted from P. crispum reference materials generated a large fragment around 219 bp and a small fragment around 62 bp, whereas DNA from A. graveolens yielded the same 219 bp large fragment and another distinctive small fragment with size around 193 bp. Although primer dimers were observed in some cases, the region between large and small fragments is relative clean. Based on the size difference of small fragments, P. crispum reference material can be easily distinguished from reference material of A. graveolens in the test scope.

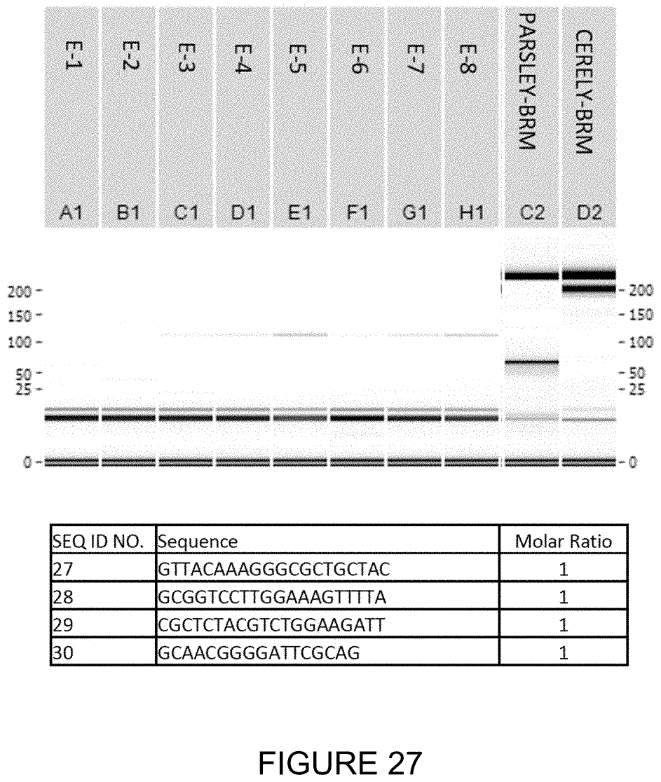

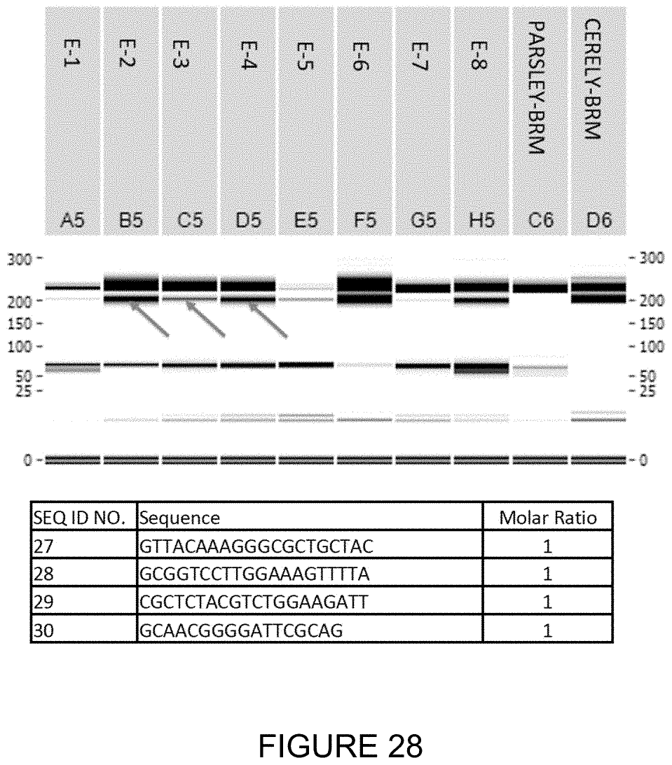

Although the low PCR cycle condition works for parsley and celery botanical reference materials that contains high quality DNA, it failed to achieve the amplification for any of the control or diagnostic bands in Parsley extracts due to DNA degradation in processed materials (FIG. 27). As a result, higher PCR cycles are required to ascertain potential control and diagnostic bands. When increasing PCR cycle number from 28 to 40, both the parsley relevant diagnostic fragment and control fragment became visible in the parsley extract samples. Thus, the current assay was able to retrieve degraded DNA from highly processed P. crispum products. However, the other 193 bp inner band, indicating the presence of A. graveolens was also visible, which may confound the result interpretation (arrows in FIG. 28). Not only was non-specific amplification observed, the band intensity of the diagnostic band in parsley botanical reference materials (BRM) also decreased.

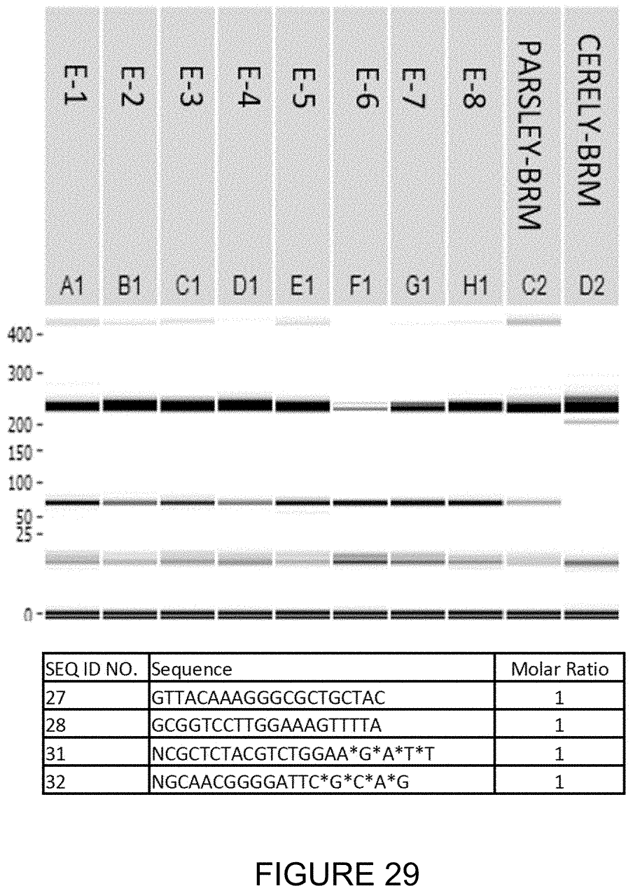

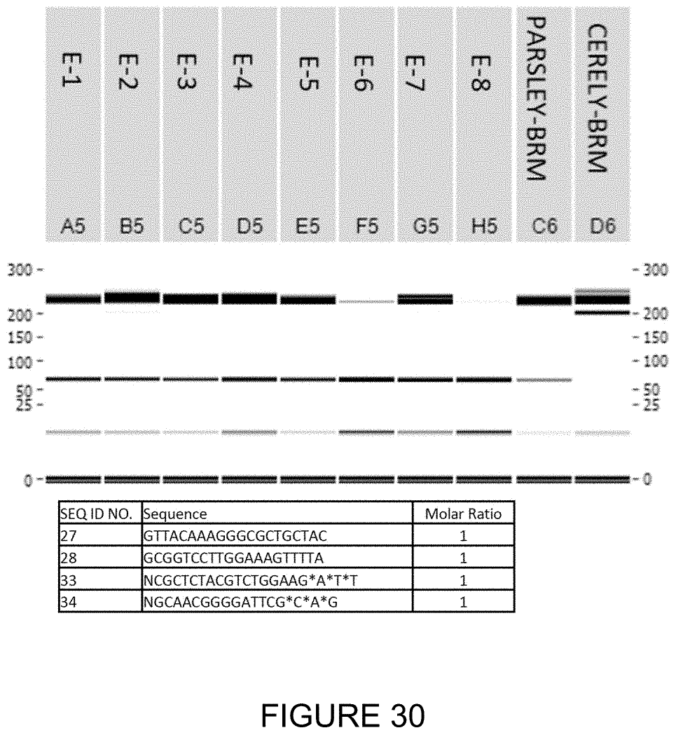

To maintain a good sensitivity and specificity in P. crispum tetra-primer ARMS-PCR in high PCR cycle numbers, both 5′ end random nucleotide and 3′ terminus phosphorothioate linkage modifications were incorporated into inner primers (SEQ ID NOs: 29-30 were replaced with SEQ ID NOs: 31-32 or 33-34). The result indicates that with modified inner primers, a universal assay condition can be applied with acceptable sensitivity and specificity to DNA extracted from eight batch parsley extracts (FIG. 29). Electrophoresis results show that primer modification can indeed prevent the non-specific amplification. The four-base phosphorothioate linkage modification (SEQ ID NOs: 31-32) on the 3′ terminus eliminated the amplification of 193 bp celery specific band, which is previously observed using regular inner primers. This modification also resulting a balanced amplification between species specific diagnostic bands in parsley and celery BRMs. In addition, three-base phosphorothioate linkage modification (SEQ ID NOs: 33-34) were also tested using the same experimental conditions and system as described below. The capillary electrophoresis result in FIG. 30 showed a more robust amplification of diagnostic bands. However, the 193 bp celery specific band was observed in some parsley extract samples. Therefore, one random nucleotide on 5′ end and four phosphorothioate linkage modifications on the 3′ terminus was the appropriate oligo modification for identifying parsley and detecting celery adulteration.

All oligos were purchased from Integrated DNA Technologies (Thermofisher, China).

Outer forward primer:
(SEQ ID NO: 27)
5′-GTTACAAAGGGCGCTGCTAC-3′,
Outer reverse primer:
(SEQ ID NO: 28)
5′-GCGGTCCTTGGAAAGTTTTA-3′
Inner forward primer (regular):
(SEQ ID NO: 29)
5′-CGCTCTACGTCTGGAAGATT-3′
Inner reverse primer (regular):
(SEQ ID NO: 30)
5′-GCAACGGGGATTCGCAG-3′.
Inner forward primer:
(SEQ ID NO: 31)
5′-NCGCTCTACGTCTGGAA*G*A*T*T-3′ (4 base 3′ modification),
Inner reverse primer:
(SEQ ID NO: 32)
5′-NGCAACGGGGATTC*G*C*A*G-3′ (4 base 3′ modification).
Inner forward primer:
(SEQ ID NO: 33)
5′-NCGCTCTACGTCTGGAAG*A*T*T-3′ (3 base 3′ modification),
Inner reverse primer:
(SEQ ID NO: 34)
5′-NGCAACGGGGATTCG*C*A*G-3′ (3 base 3′ modification).

(Note: N, random nucleotides; *, phosphorothioate bond)

The tetra-primer ARMS-PCR assay was performed in a 20 μL PCR mix, containing 10 μL AmpliTaq GoldTM 360 Master Mix, 2 μL primer mix (with 5 μM each primer), 2 μL gDNA extracted from ginseng materials, and 6 μL nuclease-free water.

The following thermal profile was used: (1) an initial step of 3 min at 95° C. and (2) 40 cycles of 30 s at 95° C., 30 s at 60° C., and 30 s at 72° C. and (3) final extension 3 min at 72° C.

### Example 4

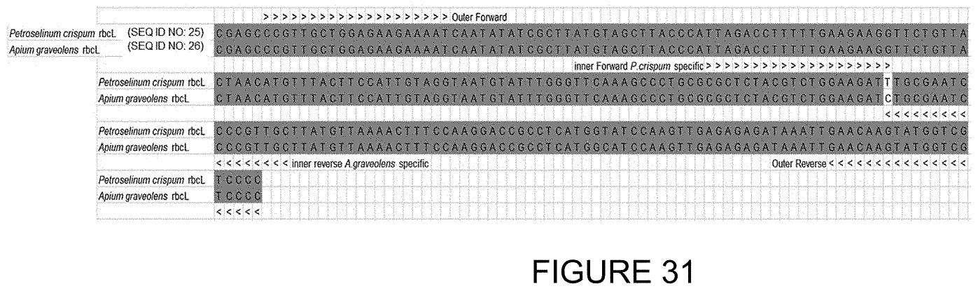

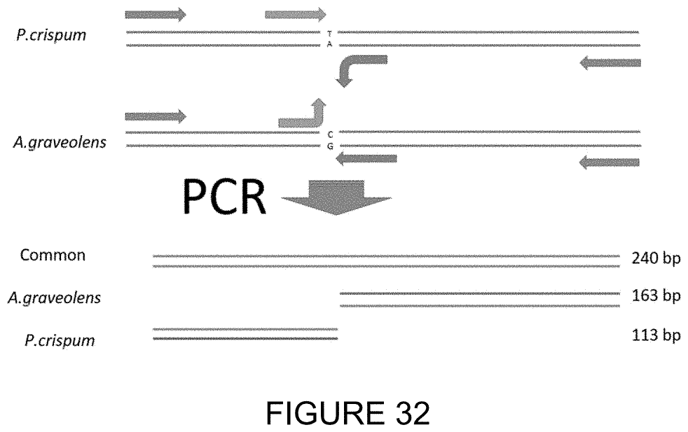

To further demonstrate the wide application of this invention, additional ARMS-PCR were designed for same SNP but using different outside primers (SEQ ID NOs: 35-36) as shown in FIG. 31. The outer primer pair amplifies a large 240 bp internal control fragment from all genomic DNA in the test scope, while the inner primers (SEQ ID NOs: 30-31) amplify two smaller diagnostic fragments 113 bp and 163 bp representing two allelic states in P. crispum and A. graveolens, respectively (FIG. 32). Based on the characteristic size of each inner fragment, the identity of P. crispum and A. graveolens can be easily resolved using DNA visualization equipment, even low-cost agarose gel electrophoresis instruments, since this new design increase the size difference between largest diagnostic band and control band.

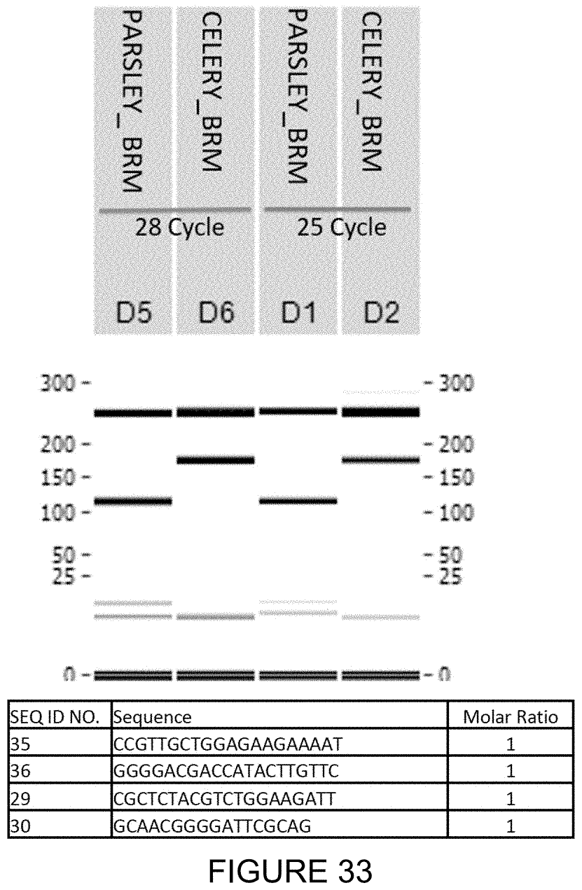

To test its feasibility of this new design in P. crispum identification and differentiation, the current tetra-primer ARMS-PCR was applied to DNA extracted from reference materials of P. crispum and A. graveolens. As shown in FIG. 33, with 25 and 28 cycles of PCR amplification, DNA extracted from P. crispum reference materials generated a large fragment around 240 bp and a small fragment around 113 bp, while DNA from A. graveolens yielded the same 240bp large fragment and another distinctive small fragment with size around 163bp.

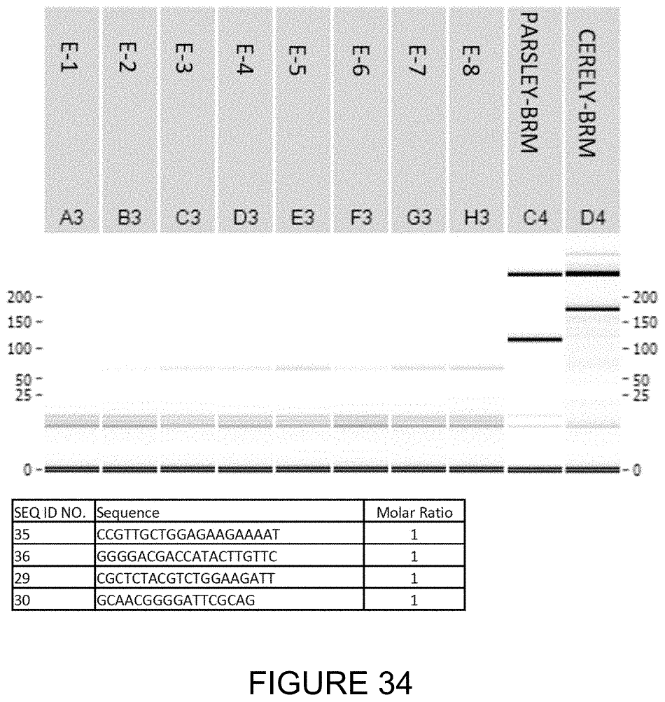

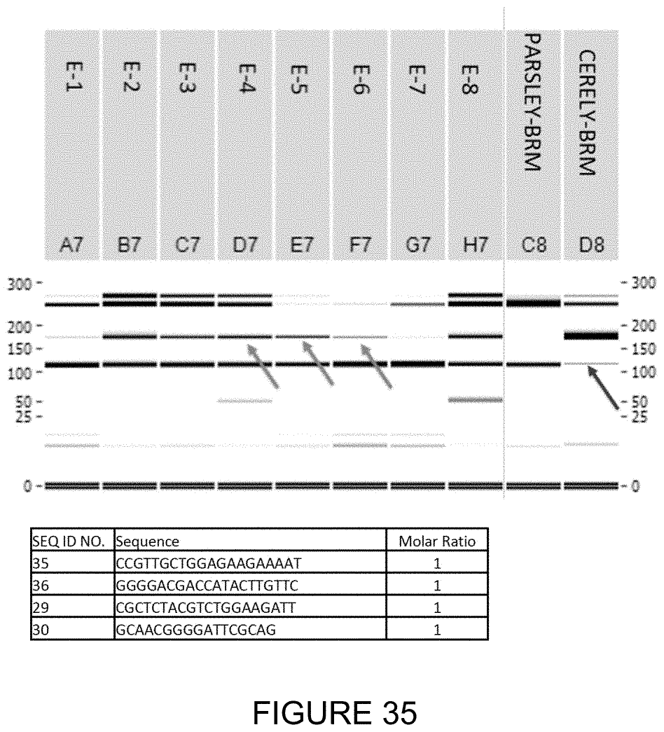

In this new design, high PCR cycle numbers (35 to 40 cycles) were still required to test processed parsley samples, since no control and diagnostic band was observed in parsley extract samples (FIG. 34). However, with the increments of PCR cycle number (40 cycles), both diagnostic bands were amplified in relatively pure parsley extract samples (left three arrows) and celery botanical reference material (most right arrow), in which only a single diagnostic band was expected (FIG. 35).

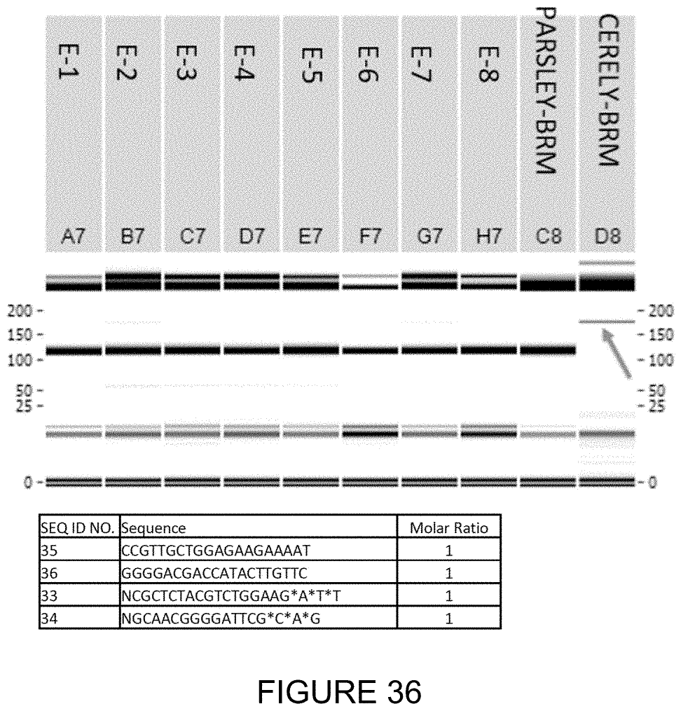

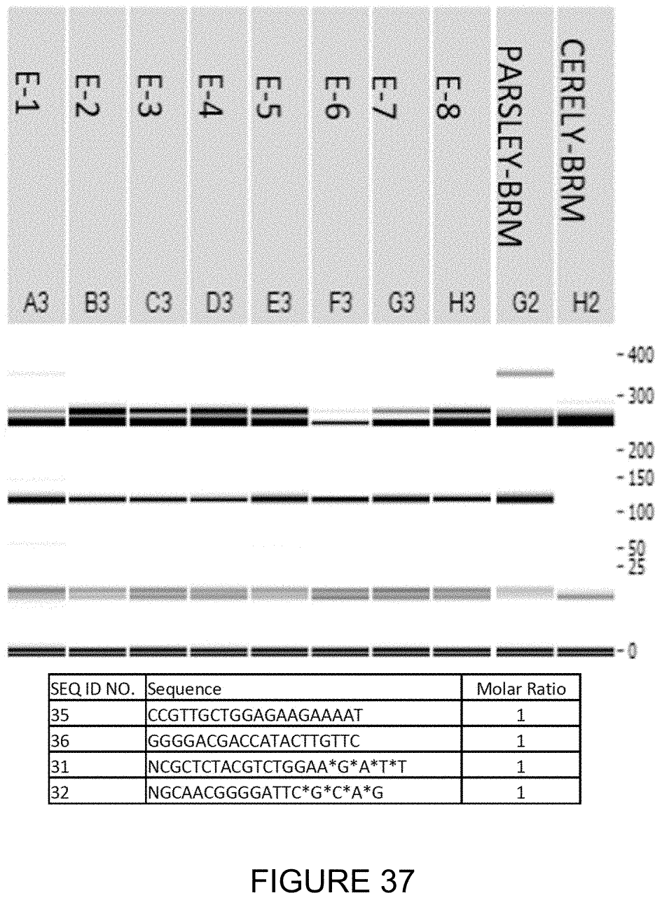

To regain specificity, inner primers with both 5′ end random nucleotide and 3′ terminus phosphorothioate linkage modifications (SEQ ID NOs: 31-32 or 33-34) were used to replace SEQ ID NOs: 29-30 again. In this case, inner primers with 3 bases of phosphorothioate linkage modifications at the 3′ terminus (SEQ ID NOs: 33-34), showed a balanced amplification between control and diagnostic bands (FIG. 36), since the amplification of 193 bp celery specific band were significantly inhibited by inner primers with 4 bases (SEQ ID NOs: 31-32) (FIG. 37, missing 163 bp band in celery-BRM lane). Therefore, one random nucleotide on 5′ end and three phosphorothioate linkage modifications on the 3′ terminus was the appropriate oligo modification in this particular ARMS-PCR design. In addition, this scheme shows that the amplification efficiency is different in different amplification regions, and it is feasible to obtain the desired amplification results by optimizing the number of primer base modifications.

All oligos were purchased from Integrated DNA Technologies (Thermofisher, China).

Outer forward primer:
(SEQ ID NO: 35)
5′-CCGTTGCTGGAGAAGAAAAT-3′,
Outer reverse primer:
(SEQ ID NO: 36)
5′-GGGGACGACCATACTTGTTC-3′,
(Note: N, random nucleotides; *, phosphorothioate bond)

The tetra-primer ARMS-PCR assay was performed in a 20 μL PCR mix, containing 10 μL AmpliTaq GoldTM 360 Master Mix, 2 μL primer mix (with 5 μM each primer), 2 μL gDNA extracted from ginseng materials, and 6 μL nuclease-free water.

The following thermal profile was used: (1) an initial step of 3 min at 95° C. and (2) 40 cycles of 30 s at 95° C., 30 s at 60° C., and 30 s at 72° C. and (3) final extension 3 min at 72° C.

The disclosure is generally described herein using affirmative language to describe the numerous embodiments. The disclosure also includes embodiments in which subject matter is excluded, in full or in part, such as substances or materials, method steps and conditions, protocols, or procedures.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

SEQUENCES

SEQ ID NO: 1 (outer F)-TCACCCCATACATAGTCTGATAGTTC
SEQ ID NO: 2 (outer R)-GAGTCAAATGGGCTTTTTGG
SEQ ID NO: 3 (inner F1)-GTCAATACCGGCAACAATGAAATTTT
SEQ ID NO: 4 (inner R1)-GTCGACGGATTTTCCTCTTACTAT
SEQ ID NO: 5 (inner F2)-GTCAATACCGGCAACAATGAAATCTT
SEQ ID NO: 6 (inner R2)-GTCGACGGATTTTCCTCTTACCAT
SEQ ID NO: 7 (inner F3)-NGTCAATACCGGCAACAATGAAATTTT
N is A or G or C or T
SEQ ID NO: 8 (inner R3)-NGTCGACGGATTTTCCTCTTACTAT
N is A or G or C or T
SEQ ID NO: 9 (inner F8)-VGTCAATACCGGCAACAATGAAATTTT
V is A or G or C
SEQ ID NO: 10 (inner R8)-BGTCGACGGATTTTCCTCTTACTAT
B is G or C or T
SEQ ID NO: 11 (inner F9)-GTCAATADCGGCAACAATGAAATTTT
D is A or G or T
SEQ ID NO: 12 (inner R9)-GTCGACGHATTTTCCTCTTACTAT
H is A or C or T
SEQ ID NO: 13 (inner R4)-NGTCGACGHATTTTCCTCTTACTGT
N is A or G or C or T; H is A or C or T
SEQ ID NO: 14 (inner R5)-NGTCGACGHATTTTCCTCTTACTCT
N is A or G or C or T; H is A or C or T
SEQ ID NO: 15 (inner R6)-GTCGACGHATTTTCCTCTTACTTT
H is A or C or T
SEQ ID NO: 16 (inner F7)-NGTCAATACCGGCAACAATGAAA*T*T*T*T
N is A or G or C or T; * is phosphorothioate bond
SEQ ID NO: 17 (inner R7)-NGTCGACGGATTTTCCTCTTA*C*T*A*T
N is A or G or C or T; * is phosphorothioate bond
SEQ ID NO: 18 (P. ginseng consensus)-
TCACCCCATACATAGTCTGATAGTTCTTTTGAAGAACTGATTAATCGGACGAGAA
TAAAGATAGAGTCCCATTCTACATGTCAATACCGGCAACAATGAAATTTTTAGTA
AGAGGAAAATCCGTCGACTTTAAAATCGTGAGGGTTCAAGTCCCTCTATCCCCAA
AAAGCCCATTTGACTC
SEQ ID NO: 19 (P. quinquefolius consensus)-
TCACCCCATACATAGTCTGATAGTTCTTTTGAAGAACTGATTAATCGGACGAGAA
TAAAGATAGAGTCCCATTCTACATGTCAATACCGGCAACAATGAAATTTATAGTA
AGAGGAAAATCCGTCGACTTTAAAATCGTGAGGGTTCAAGTCCCTCTATCCCCAA
AAAGCCCATTTGACTC
SEQ ID NO: 20 (P. notoginseng consensus)-
TCACCCCATACATAGTCTGATAGTTCTTTTGAAGAACTGATTAATCGGACGAGAA
TAAAGATAGAGTCCCATTCTACATGTCAATACCGGCAACAATGAAATTTATAGTA
AGAGGAAAATCCGTCGACTTTAAAATCGTGAGGGTTCAAGTCCCTCTATCCCCAA
AAAGCCCATTTGACTC
SEQ ID NO: 21 (P. japonicus consensus)-
TCACCCCATACATAGTCTGATAGTTCTTTTGAAGAACTGATTAATCGGACGAGAA
TAAAGATAGAGTCCCATTCTACATGTCAATACCGGCAACAATGAAATTTATAGTA
AGAGGAAAATCCGTCGACTTTAAAATCGTGAGGGTTCAAGTCCCTCTATCCCCAA
AAAGCCCATTTGACTC
SEQ ID NO: 22 (P. senticosus consensus)-
TCACCCCATACATAGTCTGATAGTTCTTTTGAAGAACTGATTAATCGGACGAGAA
TAAAGATAGAGTCCCATTCTACATGTCAATACCGGCAACAATGAAATTTATAGTA
AGAGGAAAATCCGTCGACTTTAAAATCGTGAGGGTTCAAGTCCCTCTATCCCCAA
AAAGCCCATTTGACTC
SEQ ID NO: 23 (Petroselinum crispum rbeL)-
TCTTCTACTGGTACATGGACCACTGTGTGGACCGATGGACTTACCAGTCTTGATC
GTTACAAAGGGCGCTGCTACGGAATCGAGCCCGTTGCTGGAGAAGAAAATCAAT
ATATCGCTTATGTAGCTTACCCATTAGACCTTTTTGAAGAAGGTTCTGTTACTAAC
ATGTTTACTTCCATTGTAGGTAATGTAATTTGGGTTCAAAGCCCTGCGCGCTCTA
CGTCTGGAAGATTTGCGAATCCCCGTTGCTTATGTTAAAACTTTCCAAGGACCGC
CTCATGGTATCCAAGTTGAGAGAGATAAATTGAACAAGTATGGTCG
SEQ ID NO: 24 (Apium graveolens rbeL)-
TCTTCTACTGGTACATGGACCACTGTGTGGACCGATGGACTTACCAGTCTTGATC
GTTACAAAGGGCGCTGCTACGGAATCGAGCCCGTTGCTGGAGAAGAAAATCAAT
ATATCGCTTATGTAGCTTACCCATTAGACCTTTTTGAAGAAGGTTCTGTTACTAAC
ATGTTTACTTCCATTGTAGGTAATGTAATTTGGGTTCAAAGCCCTGCGCGCTCTA
CGTCTGGAAGATCTGCGAATCCCCGTTGCTTATGTTAAAACTTTCCAAGGACCGC
CTCATGGTATCCAAGTTGAGAGAGATAAATTGAACAAGTATGGTCG
SEQ ID NO: 25 (Petroselinum crispum rbeL)-
CGAGCCCGTTGCTGGAGAAGAAAATCAATATATCGCTTATGTAGCTTACCCATTA
GACCTTTTTGAAGAAGGTTCTGTTACTAACATGTTTACTTCCATTGTAGGTAATGT
ATTTGGGTTCAAAGCCCTGCGCGCTCTACGTCTGGAAGATTTGCGAATCCCCGTT
GCTTATGTTAAAACTTTCCAAGGACCGCCTCATGGTATCCAAGTTGAGAGAGATA
AATTGAACAAGTATGGTCGTCCCC
SEQ ID NO: 26 (Apium graveolens rbeL)-
CGAGCCCGTTGCTGGAGAAGAAAATCAATATATCGCTTATGTAGCTTACCCATTA
GACCTTTTTGAAGAAGGTTCTGTTACTAACATGTTTACTTCCATTGTAGGTAATGT
ATTTGGGTTCAAAGCCCTGCGCGCTCTACGTCTGGAAGATCTGCGAATCCCCGTT
GCTTATGTTAAAACTTTCCAAGGACCGCCTCATGGTATCCAAGTTGAGAGAGATA
AATTGAACAAGTATGGTCGTCCCC
SEQ ID NO: 27 (outer forward primer)-GTTACAAAGGGCGCTGCTAC
SEQ ID NO: 28 (outer reverse primer)-GCGGTCCTTGGAAAGTTTTA
SEQ ID NO: 29 (inner forward primer regular)-CGCTCTACGTCTGGAAGATT
SEQ ID NO: 30 (inner reverse primer regular)-GCAACGGGGATTCGCAG
SEQ ID NO: 31 (inner forward primer)-NCGCTCTACGTCTGGAA*G*A*T*T
N is A or G or C or T; * is phosphorothioate bond
SEQ ID NO: 32 (inner reverse primer)-NGCAACGGGGATTC*G*C* A*G
N is A or G or C or T; * is phosphorothioate bond
SEQ ID NO: 33 (inner forward primer)-NCGCTCTACGTCTGGAAG*A*T*T
N is A or G or C or T; * is phosphorothioate bond
SEQ ID NO: 34 (inner reverse primer)-NGCAACGGGGATTCG*C*A*G
N is A or G or C or T; * is phosphorothioate bond
SEQ ID NO: 35 (outer forward primer)-CCGTTGCTGGAGAAGAAAAT
SEQ ID NO: 36 (outer reverse primer)-GGGGACGACCATACTTGTTC
SEQ ID NO: 37-AGTCGACGGATTTTCCTCTTACTAT
SEQ ID NO: 38-CGTCGACGGATTTTCCTCTTACTAT
SEQ ID NO: 39-GGTCGACGGATTTTCCTCTTACTAT
SEQ ID NO: 40-TGTCGACGGATTTTCCTCTTACTAT

### REFERENCES

The disclosure of each reference set forth herein is incorporated herein by reference in its entirety.
Abu Al-Soud, W., & Radstrom, P. (1998). Capacity of nine thermostable DNA polymerases To mediate DNA amplification in the presence of PCR-inhibiting samples. Appl Environ Microbiol, 64(10), 3748-3753.
Arulandhu, A. J., Staats, M., Hagelaar, R., Voorhuijzen, M. M., Prins, T. W., Scholtens, I., Kok, E. (2017). Development and validation of a multi-locus DNA metabarcoding method to identify endangered species in complex samples. GigaScience, 6(10).
Eom, S. H., Wang, J., & Steitz, T. A. (1996). Structure of Taq polymerase with DNA at the polymerase active site. Nature, 382(6588), 278-281.
Heissl, A., Arbeithuber, B., & Tiemann-Boege, I. (2017). High-Throughput Genotyping with TaqMan Allelic Discrimination and Allele-Specific Genotyping Assays. Methods Mol Biol, 1492, 29-57.
Huang, M. M., Arnheim, N., & Goodman, M. F. (1992). Extension of base mispairs by Taq DNA polymerase: implications for single nucleotide discrimination in PCR. Nucleic acids research, 20(17), 4567-4573.
Lee, S. M., Bae, B.-S., Park, H.-W., Ahn, N.-G., Cho, B.-G., Cho, Y.-L., & Kwak, Y.-S. (2015). Characterization of Korean Red Ginseng (Panax ginseng Meyer): History, preparation method, and chemical composition. Journal of ginseng research, 39(4), 384-391.
Li, Y., Mitaxov, V., & Waksman, G. (1999). Structure-based design of Taq DNA polymerases with improved properties of dideoxynucleotide incorporation. Proceedings of the National Academy of Sciences of the United States of America, 96(17), 9491-9496.
Little, S. (1995). Amplification-refractory mutation system (ARMS) analysis of point mutations. Current protocols in human genetics, 7(1), 9.8. 1-9.8. 12.
Little, S. (2001). Amplification-refractory mutation system (ARMS) analysis of point mutations. Current protocols in human genetics, Unit 9.8.
Lu, Z., Rubinsky, M., Babajanian, S., Zhang, Y., Chang, P., & Swanson, G. (2018). Visualization of DNA in highly processed botanical materials. Food chemistry, 245, 1042-1051.
Porebski, S., Bailey, L. G., & Baum, B. R. (1997). Modification of a CTAB DNA extraction protocol for plants containing high polysaccharide and polyphenol components. Plant Molecular Biology Reporter, 15(1), 8-15.
Quan, Z., Yang, Z., Chua, T., Li, L., Zhang, Y., Babajanian, S., . . . Lu, Z. (2020). Development and validation of a probe-based qPCR method to prevent parsley leaf material misidentification. Fitoterapia, 146, 104666.
Ragupathy, S., Faller, A. C., Shanmughanandhan, D., Kesanakurti, P., Shaanker, R. U., Ravikanth, G., . . . Newmaster, S. (2019). Exploring DNA quantity and quality from raw materials to botanical extracts. Heliyon, 5(6), e01935.
Ruhsam, M., & Hollingsworth, P. M. (2018). Authentication of Eleutherococcus and Rhodiola herbal supplement products in the United Kingdom. Journal of Pharmaceutical and Biomedical Analysis, 149, 403-409.
Shatleh-Rantisi, D., Tamimi, A., & Ashhab, Y. (2020). Improving sensitivity of single tube nested PCR to detect fastidious microorganisms. Heliyon, 6(1), e03246.
Ye, S., Dhillon, S., Ke, X., Collins, A. R., & Day, I. N. (2001). An efficient procedure for genotyping single nucleotide polymorphisms. Nucleic acids research, 29(17), E88-e88.

What is claimed is:

1. A method for identifying processed botanical material, the method comprising:
extracting genomic plant DNA from the processed botanical material, wherein the processed botanical material contains a target species and an optional non-target species;
amplifying the extracted genomic plant DNA using tetra-primer amplification refractory mutation system polymerase chain reaction (ARMS-PCR);
identifying a PCR amplicon amplified from the target species and optionally another PCR amplicon amplified from the non-target species;
thereby identifying the processed botanical material.

2. The method of claim 1, further comprising detecting adulterant in the material.

3. The method of claim 1, wherein the botanical material is Panax ginseng, Panax quinquefolius, Panax notoginseng, Panax japonicas, Eleutherococcus senticosus, or Petroselinum crispum.

4. The method of claim 1, wherein the tetra-primer ARMS-PCR includes a pair of inner primers and a pair of outer primers.

5. The method of claim 4, wherein one or both inner primers of the pair of inner primers have a 5′ end random nucleic acid modification and/or a 3′ end phosphorothioate bond modification.

6. The method of claim 4, wherein one or both inner primers of the pair of inner primers have 1-9 3′ end phosphorothioate bond modifications.

7. The method of claim 4, wherein one or both inner primers of the pair of inner primers have 4 consecutive 3′ end phosphorothioate bond modifications.

8. The method of claim 4, wherein the botanical is ginseng, and wherein the pair of inner primers comprises an inner forward primer having a sequence as set forth in SEQ ID NO: 12 and comprises an inner reverse primer having a sequence as set forth in SEQ ID NO: 13.

9. The method of claim 4, wherein the botanical is parsley, and wherein the pair of inner primers comprises an inner forward primer having a sequence as set forth in SEQ ID NO: 31 or 33 and comprises an inner reverse primer having a sequence as set forth in SEQ ID NO: 32 or 34.

10. The method of claim 1, wherein the processed botanical material is a supplement, powder, or extract.

11. A multiplex PCR system for identifying processed botanical material, wherein the processed botanical material comprises a target species and/or a closely related non-target species, said system comprises:
an inner forward primer and an inner reverse primer, wherein a 3′ terminus of the inner forward primer comprises a sequence that is complementary to a sequence specific to the target species, and wherein a 3′ terminus of the inner reverse primer comprises a sequence that is complementary to a sequence specific to the non-target species, or vice versa; and
an outer primer pair consisting of an outer forward primer and an outer reverse primer.

12. The system of claim 11, wherein the sequence specific to the target species and the sequence specific to the non-target species differ by a single base or by a deletion.

13. The system of claim 11, wherein the processed botanical material comprises an adulterant.

14. The system of claim 11, wherein the inner forward primer and/or inner reverse primer have a 5′ end random nucleic acid modification, a 3′ end phosphorothioate bonds modification, or both.

15. The system of claim 11, wherein the inner forward primer and/or inner reverse primer have 1-9 3′ end phosphorothioate bond modifications.

16. The system of claim 11, wherein the inner forward primer and/or inner reverse primer have 4 consecutive 3′ end phosphorothioate bond modifications.

17. The system of claim 11, further comprising a DNA polymerase that lacks 3′−>5′ exonuclease activity

18. The system of claim 17, wherein the DNA polymerase is a Taq DNA polymerase.

19. The system of claim 11, wherein the processed botanical material is a market ginseng root material.

20. The system of claim 11, wherein the target species is P. ginseng or Petroselinum crispum.

21. The system of claim 11, wherein the non-target species is P. quinquefolius, P. notoginseng, P. japonicus, E. senticosus, or Apium graveolens

22. The system of claim 11, wherein the inner forward primer comprises a sequence as set forth in SEQ ID NO: 12 and the inner reverse primer comprises a sequence as set forth in SEQ ID NO: 13.

23. The system of claim 11, wherein the inner forward primer comprises a sequence as set forth in SEQ ID NO: 31 or 33 and the inner reverse primer comprises a sequence as set forth in SEQ ID NO: 32 or 34.


SEQUENCE LISTING (40 sequences)

SEQ ID NO: 1 - DNA, 26 bp, synthetic construct
  source 1..26: mol_type = other DNA
tcaccccatacatagtctgatagttc

SEQ ID NO: 2 - DNA, 20 bp, synthetic construct
  source 1..20: mol_type = other DNA
gagtcaaatgggctttttgg

SEQ ID NO: 3 - DNA, 26 bp, synthetic construct
  source 1..26: mol_type = other DNA
gtcaataccggcaacaatgaaatttt

SEQ ID NO: 4 - DNA, 24 bp, synthetic construct
  source 1..24: mol_type = other DNA
gtcgacggattttcctcttactat

SEQ ID NO: 5 - DNA, 26 bp, synthetic construct
  source 1..26: mol_type = other DNA
gtcaataccggcaacaatgaaatctt

SEQ ID NO: 6 - DNA, 24 bp, synthetic construct
  source 1..24: mol_type = other DNA
gtcgacggattttcctcttaccat

SEQ ID NO: 7 - DNA, 27 bp, synthetic construct
  source 1..27: mol_type = other DNA
ngtcaataccggcaacaatgaaatttt

SEQ ID NO: 8 - DNA, 25 bp, synthetic construct
  source 1..25: mol_type = other DNA
ngtcgacggattttcctcttactat

SEQ ID NO: 9 - DNA, 27 bp, synthetic construct
  source 1..27: mol_type = other DNA
vgtcaataccggcaacaatgaaatttt

SEQ ID NO: 10 - DNA, 25 bp, synthetic construct
  source 1..25: mol_type = other DNA
bgtcgacggattttcctcttactat

SEQ ID NO: 11 - DNA, 26 bp, synthetic construct
  source 1..26: mol_type = other DNA
gtcaatadcggcaacaatgaaatttt

SEQ ID NO: 12 - DNA, 24 bp, synthetic construct
  source 1..24: mol_type = other DNA
gtcgacghattttcctcttactat

SEQ ID NO: 13 - DNA, 25 bp, synthetic construct
  source 1..25: mol_type = other DNA
ngtcgacghattttcctcttactgt

SEQ ID NO: 14 - DNA, 25 bp, synthetic construct
  source 1..25: mol_type = other DNA
ngtcgacghattttcctcttactct

SEQ ID NO: 15 - DNA, 24 bp, synthetic construct
  source 1..24: mol_type = other DNA
gtcgacghattttcctcttacttt

SEQ ID NO: 16 - DNA, 27 bp, synthetic construct
  source 1..27: mol_type = other DNA
  modified_base 23..27: mod_base = OTHER; note = phosphothioate bond
ngtcaataccggcaacaatgaaatttt

SEQ ID NO: 17 - DNA, 25 bp, synthetic construct
  source 1..25: mol_type = other DNA
  modified_base 21..25: mod_base = OTHER; note = phosphothioate bond
ngtcgacggattttcctcttactat

SEQ ID NO: 18 - DNA, 181 bp, synthetic construct
  source 1..181: mol_type = other DNA
tcaccccatacatagtctgatagttcttttgaagaactgattaatcggacgagaataaagatagagtcccattctacatgtcaataccggcaacaatgaaatttttagtaagaggaaaatccgtcgactttaaaatcgtgagggttcaagtccctctatccccaaaaagcccatttgactc

SEQ ID NO: 19 - DNA, 181 bp, synthetic construct
  source 1..181: mol_type = other DNA
tcaccccatacatagtctgatagttcttttgaagaactgattaatcggacgagaataaagatagagtcccattctacatgtcaataccggcaacaatgaaatttatagtaagaggaaaatccgtcgactttaaaatcgtgagggttcaagtccctctatccccaaaaagcccatttgactc

SEQ ID NO: 20 - DNA, 181 bp, synthetic construct; identical to SEQ ID NO: 19
  source 1..181: mol_type = other DNA

SEQ ID NO: 21 - DNA, 181 bp, synthetic construct; identical to SEQ ID NO: 19
  source 1..181: mol_type = other DNA

SEQ ID NO: 22 - DNA, 181 bp, synthetic construct; identical to SEQ ID NO: 19
  source 1..181: mol_type = other DNA

SEQ ID NO: 23 - DNA, 321 bp, synthetic construct
  source 1..321: mol_type = other DNA
tcttctactggtacatggaccactgtgtggaccgatggacttaccagtcttgatcgttacaaagggcgctgctacggaatcgagcccgttgctggagaagaaaatcaatatatcgcttatgtagcttacccattagacctttttgaagaaggttctgttactaacatgtttacttccattgtaggtaatgtaatttgggttcaaagccctgcgcgctctacgtctggaagatttgcgaatccccgttgcttatgttaaaactttccaaggaccgcctcatggtatccaagttgagagagataaattgaacaagtatggtcg

SEQ ID NO: 24 - DNA, 321 bp, synthetic construct
  source 1..321: mol_type = other DNA
tcttctactggtacatggaccactgtgtggaccgatggacttaccagtcttgatcgttacaaagggcgctgctacggaatcgagcccgttgctggagaagaaaatcaatatatcgcttatgtagcttacccattagacctttttgaagaaggttctgttactaacatgtttacttccattgtaggtaatgtaatttgggttcaaagccctgcgcgctctacgtctggaagatctgcgaatccccgttgcttatgttaaaactttccaaggaccgcctcatggtatccaagttgagagagataaattgaacaagtatggtcg

SEQ ID NO: 25 - DNA, 245 bp, synthetic construct
  source 1..245: mol_type = other DNA
cgagcccgttgctggagaagaaaatcaatatatcgcttatgtagcttacccattagacctttttgaagaaggttctgttactaacatgtttacttccattgtaggtaatgtatttgggttcaaagccctgcgcgctctacgtctggaagatttgcgaatccccgttgcttatgttaaaactttccaaggaccgcctcatggtatccaagttgagagagataaattgaacaagtatggtcgtcccc

SEQ ID NO: 26 - DNA, 245 bp, synthetic construct
  source 1..245: mol_type = other DNA
cgagcccgttgctggagaagaaaatcaatatatcgcttatgtagcttacccattagacctttttgaagaaggttctgttactaacatgtttacttccattgtaggtaatgtatttgggttcaaagccctgcgcgctctacgtctggaagatctgcgaatccccgttgcttatgttaaaactttccaaggaccgcctcatggtatccaagttgagagagataaattgaacaagtatggtcgtcccc

SEQ ID NO: 27 - DNA, 20 bp, synthetic construct
  source 1..20: mol_type = other DNA
gttacaaagggcgctgctac

SEQ ID NO: 28 - DNA, 20 bp, synthetic construct
  source 1..20: mol_type = other DNA
gcggtccttggaaagtttta

SEQ ID NO: 29 - DNA, 19 bp, synthetic construct
  source 1..19: mol_type = other DNA
cgctctacgtctggaagat

SEQ ID NO: 30 - DNA, 17 bp, synthetic construct
  source 1..17: mol_type = other DNA
gcaacggggattcgcag

SEQ ID NO: 31 - DNA, 21 bp, synthetic construct
  source 1..21: mol_type = other DNA
  modified_base 17..21: mod_base = OTHER; note = phosphothioate bond
ncgctctacgtctggaagatt

SEQ ID NO: 32 - DNA, 18 bp, synthetic construct
  source 1..18: mol_type = other DNA
  modified_base 14..18: mod_base = OTHER; note = phosphothioate bond
ngcaacggggattcgcag

SEQ ID NO: 33 - DNA, 21 bp, synthetic construct
  source 1..21: mol_type = other DNA
  modified_base 18..21: mod_base = OTHER; note = phosphothioate bond
ncgctctacgtctggaagatt

SEQ ID NO: 34 - DNA, 18 bp, synthetic construct
  source 1..18: mol_type = other DNA
  modified_base 15..18: mod_base = OTHER; note = phosphothioate bond
ngcaacggggattcgcag

SEQ ID NO: 35 - DNA, 20 bp, synthetic construct
  source 1..20: mol_type = other DNA
ccgttgctggagaagaaaat

SEQ ID NO: 36 - DNA, 20 bp, synthetic construct
  source 1..20: mol_type = other DNA
ggggacgaccatacttgttc

SEQ ID NO: 37 - DNA, 25 bp, synthetic construct
  source 1..25: mol_type = other DNA
agtcgacggattttcctcttactat

SEQ ID NO: 38 - DNA, 25 bp, synthetic construct
  source 1..25: mol_type = other DNA
cgtcgacggattttcctcttactat

SEQ ID NO: 39 - DNA, 25 bp, synthetic construct
  source 1..25: mol_type = other DNA
ggtcgacggattttcctcttactat

SEQ ID NO: 40 - DNA, 25 bp, synthetic construct
  source 1..25: mol_type = other DNA
tgtcgacggattttcctcttactat